(19) United States
(12) Patent Application Publication
(10) Pub. No.: US 2023/0330118 A1
(43) Pub. Date: Oct. 19, 2023

(54) 2-DEOXY-D-GLUCOSE FOR PREVENTION AND TREATMENT OF A VIRAL DISEASE, IN PARTICULAR OF COVID-19

(71) Applicant: EXPLORATION INVEST PTE LTD, Singapore (SG)

(72) Inventors: Piotr RIESKE, Buochs, NW (CH); Jerzy GUBERNATOR, Buochs, NW (CH); Helga KOPPER, Ebenthal (AT)

(21) Appl. No.: 17/995,919

(22) PCT Filed: Apr. 11, 2021
(86) PCT No.: PCT/IB2021/052986

§ 371 Date: Oct. 10, 2022

(30) Foreign Application Priority Data
Apr. 11, 2020 (SG) 10202003317P
Apr. 14, 2020 (SG) 10202003412U
Apr. 14, 2020 (SG) 10202003413P
Jan. 25, 2021 (WO) PCT/EP2021/051646

(51) Int. Cl. A61K 31/7004; A61K 31/7056; A61K 31/4745; A61K 31/4439; A61K 9/127; A61K 9/00; A61P 31/14; A61M 15/00

(52) U.S. Cl. CPC A61K 31/7004; A61K 31/7056; A61K 31/4745; A61K 31/4439; A61K 9/1277; A61K 9/0075; A61K 9/1271; A61P 31/14; A61M 15/0065

(57) ABSTRACT

A method of applying a substance to a human body can include providing the substance, and delivering the substance in the form of aerosol particles or powder particles to the nose or mouth of a person, wherein the particles comprise at least one active ingredient out of the group comprising ribavirin, emetine, 2-DG and NMS-873.

### TECHNICAL FIELD

The present application relates to the use of 2-DG (2-deoxy-D-glucose) for the prevention and/or the treatment of a viral disease, in particular a disease caused by an enveloped virus such as Coronaviruses that comprise a glycosylated spike protein projecting outwards from the viral envelopes.

In particular the present application relates to the field of medical methods for the prevention and/or treatment of COVID-19 (Coronavirus Disease 2019), a respiratory disease caused by SARS-CoV-2 (severe acute respiratory syndrome coronavirus 2) comprising the use of 2-DG administered in proliposomes and liposomes and/or administered by inhalation.

### BACKGROUND AND SUMMARY

2-DG is a glucose analogue e.g. as a marker for glucose uptake and hexokinase activity, as an inhibitor of glucose-6-phosphate isomerase and thereby as an inhibitor of glycolysis. Medical use of 2-DG includes radioactively labelled forms 2-DG for use in diagnostic methods such as autoradiography and further includes therapeutic applications in cancer therapy. Clinical trials revealed a very high tolerance of 2-DG up to 63 mg per kg body weight and day.

Like cancer cells, also virally infected cells exhibit a high rate of glycolysis. Gualdoni, G et al. (2018) reported glycolysis inhibition by 2-deoxyglucose (2-DG) causing glucose deprivation of infected cells resulted in decreasing rhinovirus replication in vitro and inhibited infection and inflammation in a mouse model (Gualdoni, G et al. PNAS 115, E7158-E7165 (2018). Bojkova et al. reported inhibition of glycolysis on SARS-CoV-2 replication by 2-DG (Bojkova, D. et al. Nature 583, 469-472 (2020).

So far, there is no effective treatment against SARS-CoV-2 infection. There are two stages of the disease: a first one which is accompanied by viremia and a second one when the condition of some patients, although viremia is terminated, gets worse. Treatment of patients in the first of these stages has a much greater chance of success whereas in the second stage the symptoms are much more diverse and more difficult to control. The second-stage therapy, so far is rather symptomatic.

Thus, there is still a need to provide medical treatment of Covid-19, in particular preventive and therapeutic treatment for the first and the second stage of the Covid-19 disease.

Hence, it is a general object of the application to provide a preparation of 2-DG that is suitable for treatment of a viral disease caused by an enveloped viruses, in particular by SARS-CoV-2.

In order to implement these and still further objects of the application, which will become more readily apparent as the description proceeds, the use of 2-DG in medical applications of the application is described below in various aspects and embodiments:

In aspects of the application, 2-Deoxy-D-Glucose (2-DG) is provided for use in a medical method to prevent and/or treat a viral disease, in particular Covid-19. These three aspects of the application can be implemented independently of each other or in any combination unless the context clearly dictates the contrary.

In the further aspect of the application, 2-DG is provided for use in a medical method to prevent and/or to treat Covid-19, wherein 2-DG is provided as a preparation in an amount and a formulation that results in an effective tissue concentration that achieves partial or complete inhibition of glycosylation of a SARS-CoV-2 spike protein.

In an embodiment, the application provides 2-Deoxy-D-Glucose (2-DG) for use in a medical method to prevent and/or to treat a viral infection in a subject by a virus comprising a spike protein, wherein 2-DG is provided as a preparation in an amount and a formulation to tissue of a subject that results in an effective tissue concentration to partially or completely inhibit glycosylation of the spike protein.

In a further embodiment, the application provides a method of preventing and/or treating a viral infection in a subject by a virus comprising a spike protein, the method comprising administration of 2-DG to tissue of a subject in an amount and a formulation that results in an effective tissue concentration to partially or completely inhibit glycosylation of the spike protein.

In a further embodiment, 2-DG is administered with at least one of the group comprising ribavirin, emetine, and NMS-873.

The application further provides a method of preventing and/or treating a viral infection in a subject by a virus comprising a spike protein, the method comprising administration at least one of: 2-DG, ribavirin, emetine, and NMS-873 to tissue of a subject in an amount and a formulation that results in an effective tissue concentration to partially or completely inhibit glycosylation of the spike protein.

In a further embodiment, the subject is a human or an animal.

The effective tissue concentration preferably inhibits at least 30%, in particular at least 50%, 70%, 80% 90%, 95% or 99% of the glycosylation the spike protein.

The effective tissue concentration of 2-DG is preferably in a range of between approximately 0.1 mM to 25 mM.

In an embodiment, the tissue comprises respiratory tissue. More preferably, the respiratory tissue comprises epithelial cells.

In a further embodiment, the virus is enveloped. More preferably, the virus is a Coronavirus. Even more preferably, the Coronavirus is SARS-CoV-2.

In a further embodiment, the viral spike protein comprises a SARS-CoV-2 spike protein.

The viral infection is preferably in cells of the airways and respiratory tissue of a subject. More preferably, the viral infection has developed into the viral disease Covid-19.

In a further aspect of the application, 2-DG is provided for use in a medical method to prevent and/or to treat a viral disease caused by an enveloped virus comprising a spike protein, wherein 2-DG is provided as a preparation in a liposomal or a proliposomal formulation.

In a further embodiment, 2-DG is provided as a micron or a submicron particle in a preparation, wherein in particular said micron or submicron particle is a mechanically micronized particle or is a micronized particle obtained by spray drying.

In a further embodiment, 2-DG is provided as a preparation in a liposomal or a proliposomal formulation preferably achieved by spray-dying method, nebulisation method and other liposomes preparation method.

The preparation can comprise an amount of 2-DG in a range of between approximately 1% and 75% w/w of the total weight of the preparation, in particular an amount of 2-DG in a range with lower limit of approximately 10% or 20% or 30% and an upper limit of approximately 35% or 45% to 55% w/w of the total weight of the preparation, in particular between approximately 10% and 40% w/w or between approximately 15% w/w and 30% w/w of the total weight of the preparation.

The preparation preferably further comprises an excipient comprising a lipid fraction comprising or consisting of a phospholipid fraction in an amount of approximately 5% to 80% w/w, in particular approximately 15% to 50% w/w of the total weight of the preparation.

The total phospholipid fraction preferably comprises at least approximately 10% w/w up to 60% w/w, preferably in a range between approximately 20% w/w and 40% w/w most preferably in a range between approximately 30% w/w and 50% w/w of a combination of dipalmitoyl phosphatidylcholine (DPPC) and dimyristoylphosphatidylcholine (DMPC) in any weight ratio.

The preparation comprises DPPC and DMPC in a molar ratio from approximately 50:50 to 90:10, preferably a molar ratio of DPPC to DMPC from approximately 60:40 to 75:25 molar ratio, most preferably from approximately 65:35 to 71:29 molar ratio (phase transition temperature ranging from approximately 35 to 36.3° C.).

In a further embodiment, the preparation comprises a further excipient selected from the group of excipients comprising:
an amino acid, in particular leucine or glycine, in particular in an amount of 0% w/w up to approximately 80% w/w of the total weight of the preparation, more particular in an amount of approximately 10% w/w up to approximately 80% w/w, more particular in an amount of approximately 10% w/w up to 50% w/w, more particular in an amount of approximately 10% w/w up to 30% w/w;
trehalose in an amount of 0% w/w up to approximately 60% w/w of the total weight of the preparation, more particular in an amount of approximately 5% w/w up to 30% w/w;
mannitol, 0% w/w up to 60% w/w of the total weight of the preparation, more particular in an amount of 5% w/w up to 30% w/w;
propylene glycol or/and, glycerol, ethyl alcohol in the concentration range from 10 to 80% of the total weight of the liquid preparation;
one or more further phospholipid, in particular a natural or a semi-synthetic phospholipid, one or more further negatively or a positively charged phospholipid, in particular in an amount of approximately 1% up to 10% of the molar % of the phospholipid fraction, more particular in an amount of approximately 5 molar % up to 10 molar %, wherein in particular the one or more further phospholipid is in particular selected from the group comprising phosphatidylglycerol, dimyristoyl phosphatidylglycerol, dipalmitoylphosphatidylglycerol, hydrogenated soybean phosphatidylcholine (HSPC), soybean phosphatidylcholine (SPC) and wherein optionally the phospholipids comprises DPPE or DSPE with covalently attached hydrophilic polymer, in particular a PEG or polyglycerol in a molar ratio of 0 to approximately 10 molar % of the total lipid fraction, more particular in an amount of approximately 5 molar % of total lipid fraction;
sterol, in particular cholesterol in an amount of 0 molar % up to approximately 55 molar % of the total the lipid fraction, more particular in an amount of approximately 30 molar % up to 45 molar %;
nicotinic acid amide, in an amount of approximately 10% w/w up to 80% w/w of the total weight of the preparation, more particular in an amount of approximately 20 to 60% w/w of the preparation; and/or
urea in an amount of approximately 20% w/w up to 80% w/w of the total weight of the preparation, more particular in an amount of approximately 40 to 60% w/w of the preparation.

In a further embodiment, the formulation comprising:
liposome sizes ranging from approximately 30 nm to 200 nm in particular for intravenous delivery;
liposome sizes ranging from approximately 50 nm to 5 μm, in particular for pulmonary delivery;
unilamellar liposomes of sizes ranging from approximately 30 to 120 nm.

In a further embodiment:
liposome sizes range from approximately 30 nm to 200 nm in particular for intravenous delivery;
liposome sizes range from approximately 50 nm to 5 μm, in particular for pulmonary delivery;
unilamellar liposomes sizes range from approximately 30 to 120 nm.

In a further embodiment, the liposomal or a proliposomal formulation upon contact with an aqueous environment forms liposomes within a size range selected from group of size ranges comprising:
liposome sizes ranging from approximately 30 nm to 200 nm in particular for intravenous delivery;
liposome sizes ranging from approximately 50 nm to 5 μm, in particular for pulmonary delivery;
unilamellar liposomes of sizes ranging from approximately 30 to 120 nm.

In a further embodiment, the liposomes comprise an amount of encapsulated 2-DG in a range of approximately 10 mg to 1000 mg, in particular approximately 50 mg to 500 mg, preferably, approximately 100-200 mg per unit dosage. Preferably, the amount of 2-DG in the liposomal or proliposomal formulation is in a range of approximately 10 mg to 1000 mg, in particular approximately 50 mg to 500 mg, preferably, approximately 100-200 mg per unit dosage.

In a further embodiment, 2-DG is provided as a preparation in a liposomal or proliposomal slow release formulation in particular for intravenous administration, and wherein the amount of the active ingredient, 2-DG, released at the time of administration (t=0) ranges from approximately 10% to 70% w/w of the total amount of the active ingredient in the preparation and preferably ranges from approximately 30% to 50% w/w.

In a further embodiment, 2-DG is provided as a preparation in a liposomal or proliposomal slow release formulation in particular for intravenous administration, and wherein a dosage of 2-DG, in particular one unit dosage, preferably according to dosages described herein is administered at intervals between approximately once in 2 hours to 48 hours, in particular between approximately once in 4 to 24 hours or at intervals of approximately 6 or 12 hours.

In a further embodiment, the liposomal or proliposomal formulation obtained by a method of preparation selected from a group of methods comprising:
lyophilizing a liposomal formulation comprising 2-DG as active ingredient, wherein in particular the preparation
by spray drying a composition comprising 2-DG, phospholipids and optional further excipients,
wherein the optional further excipient is preferably selected from the group comprising:
   auxiliary phospholipid for spray drying selected from natural phosphatidylglycerol, DMPG, DPPG, DSPG and natural cardiolipin used at a concentration of 0 to approximately 30 mol % of the total phospholipid content; and/or
auxiliary lipids for spray drying selected from the group of sterols, in particular cholesterol,
the method of preparation of proliposomes by dehydration-rehydration a composition comprising 2-DG, phospholipids and optional excipients followed by extrusion and spray drying for the formation of unilamellar liposomes.
by nebulization of the liquid proliposomal formulation containing 2DG, lipids (phospholipids, sterols) and one of the selected solvents such like propylene glycol, glycerol, alcohol and others to form liposomes by micron solution particles upon contact with water;

In a further embodiment, 2-DG is provided as a preparation for administration by inhalation, wherein the preparation comprises particles or droplets for inhalation with a diameter of approximately 10 μm or less, in particular less than approximately 5 μm, 3 μm, 1 μm, 0.3 μm or 0.1 μm, more particular particles with a diameter in a range with a lower limit between approximately 0.1 μm and 1 μm and with an upper limit between approximately 0.5 and 5 μm.

In a further embodiment, 2-DG is provided as a dry preparation for administration by inhalation, wherein the preparation comprises a content of 2-DG as active ingredient of between approximately 5% to 80% w/w of the total dry weight of the preparation, preferably between approximately 15% to 60% w/w.

In a further embodiment, 2-DG is provided is formulated as a micron or a submicron particle or droplet for administration by a nebulizer, wherein in particular 2-DG is dissolved in an isotonic solution, in particular in approximately 0.9% saline or in water/organic solution containing lipids.

In a further embodiment, 2-DG is provided as a preparation for administration by inhalation, wherein the preparation comprises an amount of 2-DG as active ingredient per unit dosage of between approximately 0.1 mg to 20 mg, in particular between approximately 0.25 mg to 10 mg, more particularly between approximately 1 to 2 mg.

In a further embodiment, the size of the unilamellar liposomes comprise between approximately 30 nm to 250 nm.

2-DG is preferably provided as a preparation for administration by inhalation comprising lipids or surfactants comprising one or more of Tween 20, Tween 80, Pluronics, and 2-DG in solution are encapsulated within liposomes from between approximately 1 to 600 mM, and/or encapsulated in unilamellar liposomes of the size from between approximately 30-250 nm.

In a further embodiment, the application further provides a process for preparing a liposome-encapsulated pharmaceutical composition comprising at least one active ingredient selected from the group comprising ribavirin, emetine, 2-DG and NMS-873. More preferably, the liposome-encapsulated pharmaceutical composition comprises 2-DG and at least one active ingredient selected from the group comprising ribavirin, emetine, and NMS-873. Even more preferably, the active ingredient comprises 2-DG.

In a further embodiment, the application further provides a process for preparing a liposome-encapsulated pharmaceutical composition comprising 2-DG for use in a medical method to prevent and/or to treat a viral infection in a subject by a virus comprising a spike protein, wherein 2-DG is provided as a preparation in an amount and a formulation to tissue of a subject that results in an effective tissue concentration to partially or completely inhibit glycosylation of the spike protein.

In a further embodiment, the process for preparing the liposome-encapsulated pharmaceutical composition comprises the step of spray drying the liposome-encapsulated pharmaceutical composition into particles. Preferably, the spray dried particles are less than approximately 5 μm in diameter.

In the further aspect of the application, 2-DG is provided for use in a medical method to prevent and/or to treat Covid-19, wherein 2-DG is provided as a preparation for administration by inhalation, wherein in particular 2-DG is formulated as a micron or a submicron particle.

2-DG is preferably provided as a preparation for administration by inhalation as a slow release formulation, wherein the amount of the active ingredient, 2-DG, released at the time of administration (t=0) ranges from between approximately 10% to 70% w/w of the total amount of the active ingredient in the preparation and preferably ranges from between approximately 30% to 50% w/w.

2-DG is preferably provided as a preparation for administration by inhalation, wherein a dosage of 2-DG, in particular one unit dosage, preferably according to dosages described herein, is administered at intervals between approximately once in 0.5 hours to 24 hours, in particular between approximately once in 1 to 12 hours and preferably at intervals of approximately up to 2 or 4 or 6 or 8 or 10 or 12 or 24 hours.

In a further embodiment, the application provides a method of applying a substance to the body of a subject, the method comprising:
providing the substance, and
delivering the substance in the form of aerosol particles or powder particles to the nose or mouth of a subject, wherein the particles comprise at least one active ingredient selected from the group comprising ribavirin, emetine, 2-DG and NMS-873. More preferably, the particles comprise 2-DG and at least one active ingredient selected from the group comprising ribavirin, emetine, and NMS-873. Even more preferably, the active ingredient comprises 2-DG.

The particles preferably comprise a carrier material carrying the active ingredient. The carrier material preferably comprises at least one liquid selected from the group comprising: water, alcohol, propylene glycol, glycerol, liquid glucose, and/or aqueous solution. The particles preferably comprise at least a lactose and/or liposome. Delivering of the substance preferably comprises propelling the substance by means of at least one propellant comprising CFC (chlorofluorocarbon) and/or HFA (hydro-fluoroalkane).

In a further embodiment, the application pro-vides a use of a substance for inhalation, wherein the substance is provided in the form of aerosol particles or powder particles, and wherein the particles comprise at least one active ingredient selected from the group comprising ribavirin, emetine, 2-DG and NMS-873. More preferably, the particles comprise 2-DG and at least one active ingredient selected from the group comprising ribavirin, emetine, and NMS-873. Even more preferably, the active ingredient comprises 2-DG.

In a further embodiment, the application pro-vides a method of dispensing a substance, the method comprising:
providing the substance in the form of aerosol particles or powder particles,
creating an aerosol with the particles suspended in the aerosol,
creating a directed flow of aerosol such that the suspended particles move essentially along the flow direction of the aerosol, wherein the particles comprise at least one active ingredient selected from the group comprising ribavirin, emetine, 2-DG and NMS-873. More preferably, the particles comprise 2-DG and at least one active ingredient selected from the group comprising ribavirin, emetine, and NMS-873. Even more preferably, the active ingredient comprises 2-DG.

In a further embodiment, the application pro-vides a device for inhaling a substance in the form of aerosol particles or powder particles, the device comprising:
a discharge nozzle for dispensing the substance in the form of aerosol particles or powder particles,
a container for receiving and keeping the substance, and
an actuator for activating the device, the actuator being configured to release a certain amount or dose of the substance kept in the container for conveying the substance through the discharge nozzle of the device, wherein the particles comprise at least one active ingredient selected from the group comprising ribavirin, emetine, 2-DG and NMS-873. More preferably, the particles comprise 2-DG and at least one active ingredient selected from the group comprising ribavirin, emetine, and NMS-873. Even more preferably, the active ingredient comprises 2-DG.

The actuator is preferably a manual actuator which can be activated manually. The device preferably comprises a dosage valve defining the amount of the substance to be released, and wherein the actuator is configured to activate the dosage valve. The dosage valve is preferably an adjustable valve such that the dosage of the substance can be adjusted prior to dispensing the substance. Upstream to the discharge nozzle, an air flow channel for conveying the substance released by the actuator to the discharge nozzle is preferably arranged. The air flow in the air flow channel is preferably created by the user by inhaling the air while keeping the nozzle of the device in a nostril or in the mouth. The actuator is preferably further configured to provide a pressurized air flow in the airflow channel. The device preferably further comprises a reservoir with a propellant, and wherein the actuator is further configured to release the propellant such that the substance can be conveyed by droplets of propellant along the flow channel.

In a further embodiment, the application pro-vides a substance for applying to a human or animal body for the treatment of lung tissue cells by inhalation, the substance comprising at least one active ingredient selected from the group comprising ribavirin, emetine, 2-DG and NMS-873. More preferably the substance comprises 2-DG and at least one active ingredient selected from the group comprising ribavirin, emetine, and NMS-873. Even more preferably, the active ingredient comprises 2-DG.

The substance is preferably for use in the therapy of COVID-19. The substance preferably comprises non-toxic concentration of the active ingredient for preventing SARS-CoV-2 replication in human lung tissue cells or in human nasal mucosa cells.

The substance preferably comprises a carrier material carrying the active ingredient. The carrier material preferably comprises at least one liquid selected from: water, alcohol, liquid glucose, and/or aqueous solution. The substance preferably comprises lactose and/or liposome.

In a further embodiment, the application provides a pharmaceutical composition for administration to the airways of a subject, the pharmaceutical composition comprising at least one active ingredient selected from the group comprising: ribavirin, emetine, 2-DG and NMS-873. More preferably the active ingredient comprises 2-DG and at least one selected from the group comprising ribavirin, emetine, and NMS-873. Even more preferably, the active ingredient comprises 2-DG.

In a further embodiment, the application pro-vides a method of treating a subject, the method comprising the step of inhalation by a subject of a pharmaceutical composition comprising at least one active ingredient selected from the group comprising: ribavirin, emetine, 2-DG and NMS-873. More preferably the active ingredient comprises 2-DG and at least one selected from the group comprising ribavirin, emetine, and NMS-873. Even more preferably, the active ingredient comprises 2-DG.

In a further embodiment, the application pro-vides a method of producing a pharmaceutical composition for administration to the airways of a subject, the pharmaceutical composition comprising at least one active ingredient selected from the group comprising: ribavirin, emetine, 2-DG and NMS-873. More preferably the active ingredient comprises 2-DG and at least one selected from the group comprising ribavirin, emetine, and NMS-873. Even more preferably, the active ingredient comprises 2-DG.

In a further embodiment, the application pro-vides a method of producing an inhalable pharmaceutical composition for a subject, the pharmaceutical composition comprising at least one active ingredient selected from the group comprising: ribavirin, emetine, 2-DG and NMS-873. More preferably the active ingredient comprises 2-DG and at least one selected from the group comprising ribavirin, emetine, and NMS-873. Even more preferably, the active ingredient comprises 2-DG.

In a further embodiment, the application pro-vides for the manufacture of a pharmaceutical composition comprising at least one active ingredient selected from the group comprising: ribavirin, emetine, 2-DG and NMS-873, for administration to the airways of a subject. More preferably the active ingredient comprises 2-DG and at least one selected from the group comprising ribavirin, emetine, and NMS-873. Even more preferably, the active ingredient comprises 2-DG.

The application further provides a method of manufacturing a proliposome- or liposome-encapsulated pharmaceutical composition comprising 2-DG for use in a medical method to prevent and/or to treat a viral infection in a subject by a virus comprising a spike protein, wherein 2-DG is provided as a preparation in an amount and a formulation to tissue of a subject that results in an effective tissue concentration to partially or completely inhibit glycosylation of the spike protein.

In a further embodiment, the application pro-vides for the administration to the airways of a subject a pharmaceutical composition comprising at least one active ingredient selected from the group comprising: ribavirin, emetine, 2-DG and NMS-873. More preferably the active ingredient comprises 2-DG and at least one selected from the group comprising ribavirin, emetine, and NMS-873. Even more preferably, the active ingredient comprises 2-DG.

In a further embodiment, the application pro-vides for the pulmonary delivery of a pharmaceutical composition to a subject, the pharmaceutical composition comprising at least one active ingredient selected from the group comprising: ribavirin, emetine, 2-DG and NMS-873. More preferably the active ingredient comprises 2-DG and at least one selected from the group comprising ribavirin, emetine, and NMS-873. Even more preferably, the active ingredient comprises 2-DG.

In a further embodiment, the application pro-vides a use of a pharmaceutical composition comprising at least one active ingredient selected from the group comprising: ribavirin, emetine, 2-DG and NMS-873, for the treatment of a viral infection in the airways of a subject. More preferably the active ingredient comprises 2-DG and at least one selected from the group comprising ribavirin, emetine, and NMS-873. Even more preferably, the active ingredient comprises 2-DG.

In a further embodiment, the application pro-vides a use of a pharmaceutical composition comprising at least one active ingredient selected from the group comprising: ribavirin, emetine, 2-DG and NMS-873, in the manufacture of a medicament for the prevention and/or treatment of a viral infection in the airways of a subject. More preferably the active ingredient comprises 2-DG and at least one selected from the group comprising ribavirin, emetine, and NMS-873. Even more preferably, the active ingredient comprises 2-DG.

The pharmaceutical composition preferably comprises a powder or an aerosolised form. The pharmaceutical composition preferably comprises a liposome-encapsulated pharmaceutical composition. The pharmaceutical composition is preferably delivered to the respiratory tract of the subject.

Administration or delivery to the airways of a subject preferably comprises inhalation by the subject. Inhalation is preferably through the mouth and/or the nose of the subject.

An inhalation device is preferably used to dispense the pharmaceutical composition for inhalation by the subject. The inhalation device preferably comprises a pressurized meter dose inhaler (pMDIs), nebulizer, or a dry powder inhaler (DPIs). The pharmaceutical composition preferably enters cells of the respiratory tract of the subject. In further aspects of the application, a method of treating a human or non-human animal in need thereof is provided comprising the administration of 2-DG according to an embodiment of the first aspect, the second or the further aspect of the application or any combination thereof.

In further aspects of the application, a method of treating a human or non-human animal in need thereof is provided comprising the administration of 2-DG according to an embodiment of the further aspect or the second or the further aspect of the application or any combination thereof.

In further aspects of the application, a use of 2-DG for the manufacture of a medicament according to an embodiment of the further aspect or the second or the further aspect of the application or any combination thereof is provided.

In a further aspect of the application, a pharmaceutical formulation is provided comprising 2-DG according to an embodiment of the further aspect or the second or the further aspect of the application or any combination thereof is provided.

In yet further aspects of the application, a device for inhaling a preparation (substance) comprising 2-DG in the form of aerosol particles or powder particles, in particular according to an embodiment of the first or the second or the further aspect of the application or any combination thereof is provided.

In yet further aspects of the application, a kit is provided comprising at least 2-DG, wherein 2-DG is provided as a micron or a submicron particle in a preparation, wherein in particular said micron or submicron particle is preferably a mechanically micronized particle or is a micronized particle obtained by spray drying, or 2-DG is provided as a preparation in a liposomal or a proliposomal formulation, preferably obtained by spray drying. The kit preferably comprises a means for a subject to inhale the preparation comprising 2-DG. Preferably, the kit comprises a pressurized meter dose inhaler (pMDIs), nebulizer, or a dry powder inhaler (DPIs).

In the further aspect and further aspects of the application, an anti-COVID-19 method for applying a substance to a human body, use of the substance, as well as a method and device for dispensing the substance is provided. The term “substance” in the following description of the third and further aspects of the application, in particular relates to a medical preparation comprising 2-DG for use in a medical method to prevent and/or treat Covid 19—albeit some other substances of interest might be mentioned, too.

The further aspect and further aspects in particular relates to methods for applying substances to a human body, in particular the application relates to a method for applying a substance to a human body, use of the substance, as well as a method and device for dispensing the substance.

A further object of the present application is to provide an improved method for applying a substance to a human body, a novel use of the substance, as well as an improved method and device for applying the substance.

According to some embodiments, a method of applying a substance or formulation to a human body is provided. The method comprises providing the substance and delivering the substance in the form of aerosol particles or powder particles to the nose or mouth of a person or animal. In particular, these particles can be fine or sub-micron particles, such that, from the nose and mouth, they can also travel along the entire respiratory tract, in particular, to the low respiratory tract of the person. The particles comprise at least one active ingredient, in particular the particles comprise 2-DG. Some active ingredients can serve as translation inhibitors for preventing or suppressing viral replication and/or as agents for suppressing the growth and reproduction of the host cells attacked by viruses. Due to prevention of the viral replication and suppressing the growth and reproduction of the host cells, these active ingredients can serve not only as a medication against viral-infectious diseases but also as a prophylaxis for preventing a viral attack or contagion of the human body.

For example ribavirin with the molecular formula C8H12N4O5 is a nucleoside analogue and antiviral agent with an activity against hepatitis C virus. Emetine with the molecular formula C29H40N2O4 can be isolated from the root of the plant Psychotria Ipecacuanha (ipecac root) and other plants with antiemetic and anthelminthic properties and inhibits protein synthesis in eukaryotic cells by irreversibly blocking ribosome movement along the mRNA (messenger Ribonucleic acid) strand and inhibits DNA (Deoxyribonucleic acid) replication in the early S phase (Synthesis Phase) of the cell cycle. 2-DG (2-deoxyglucose), with the molecular formula C6H12O5, is a glucose molecule which has 2-hydroxyl group replaced by hydrogen. NMS-873 with the molecular formula C27H28N4O3S2 can activate protein response and modulate autophagosome maturation. In some embodiments, the substance is delivered in such a way that the concentration of the active ingredient in the body or organism remains in a non-toxic concentration range.

In some embodiments, the particles can comprise a carrier material carrying the active ingredient. The carrier material can be in particular provided in the form of a matrix in which the active ingredient resides. The carrier material can in particular facilitate the handling and dispensing of the active agent.

The carrier material may comprise a liquid out of the group comprising water, alcohol, liquid glucose, or aqueous solution. The active ingredient or agent, which may be suspended in the liquid, can be thus easily dispensed together with the liquid. The aqueous solution with 2% salt may serve as a preservation medium for the active agent until it is dispensed. In particular, the particles may be provided in a suspension or spray formulation. The liquid carrier material can facilitate quantification or dosage of the active ingredient released together with the liquid.

In some embodiments, the particles comprise at least a lactose and/or a liposome. Lactose or liposome can serve as a carrier for the active ingredient, such that they can be easily transferred or delivered to the human body. In some embodiments, the active agent is encapsulated in a liposome, in order to achieve a sustained release with a long-lasting or retarded effect of the active agent, thus increasing the duration of the desired effect. Particles may also comprise cholesterol, which stabilizes liposomes such that an even greater delay of the activation of the agent or active ingredient can be achieved.

The particles may comprise a mixture of different liposomes.

In particular, a mixture of small liposomes, with an average size of less than 100 nm and large liposomes, with an average size of more than 150 nm. By providing different liposome sizes, a desired time profile of the active agent activity can be achieved.

The method may further comprise propelling the substance by means of at least one propellant comprising CFC (chlorofluorocarbon) and/or HFA (hydrofluoroalkane). The propellant can in particular, facilitate the delivery and dosage of the active ingredients.

The amount of the active agents and the dosage may be kept in the range of 5 to 10 millimoles. By limiting the amount of the active agent in the particles, the side effects related with too high dosage of the active ingredients can be avoided. In some embodiments the liposomes have a transition temperature, from solid to liquid, in the range from 35° C. to 45° C., more specifically, between 37° C. and 40° C. degrees about 37° C. Thus, the liposomes may easier dissolve after the substance has been applied to the human body.

According to another aspect of the present application, a use of a substance for inhalation is suggested. Thereby, the substance is provided in the form of aerosol particles or powder particles, wherein the particles comprise at least one active ingredient or active agent, in particular 2-DG, and wherein the substance is delivered to the mouth or nose of a person. In particular, the substance may be, delivered to the mouth or nose of the person by means of an inhalator which can be operated personally by the user.

By delivering the substance to the nose or the mouth of the person, at least a portion of the active ingredients can arrive at inner regions of the mouth and the nose and also deeper in the respiratory tract, in particular the lungs, of the person. These active ingredients can serve as translation inhibitors for preventing or subduing viral replications in human cells. Due to prevention of the viral replication, these active ingredients can also serve as a prophylaxis for preventing pathological development if a person is exposed to a viral infection.

According to a further aspect, a method for dispensing a substance is provided. The method comprises providing the substance in the form of particles or powder, creating an aerosol with the particles suspended in the aerosol, and creating a directed flow of the aerosol such that the suspended particles move essentially along the flow direction of the aerosol, wherein the particles comprise at least one active ingredient, in particular 2-DG. In particular, the particles may be provided in the Form of compound particles comprising a matrix or carrier material and the active ingredient. The matrix or carrier material can facilitate keeping, handling and dispensing of the active agent in a controlled way. The method further comprises directing the directed flow or jet of the aerosol towards target areas of the human body for dispensing the substance. Hence, the substance with the active agent can be purposefully applied to specific areas of the human body.

According to still another aspect, a device for inhaling a substance in the form of aerosol particles or powder particles is provided. The device comprises a container for receiving and keeping the substance. The device further comprises an actuator for activating the device, the actuator is configured to release a certain amount or dose of the substance kept in the container for conveying through the discharge nozzle of the device, wherein the particles comprise at least one active ingredient, in particular 2-DG.

Thus, the substance can be dispensed in small doses, in order to keep the concentration of the active agent at a moderate level, in particular, to avoid an overdosage of the active ingredient and the side effects associated with the overdosage.

The actuator may be a manual actuator which can be activated or triggered manually. Thus, the user himself can activate the device whenever it is needed.

The device may comprise a dosage valve defining the amount of the released substance, each time when the activator is triggered, and the actuator may be configured to activate the dosage valve. By means of the dosage valve, a precise dosage of the substance, in particular, of the active agent can be achieved. The dosage valve may be an adjustable dosage valve such that the dosage of the substance can be adjusted prior to dispensing the substance.

In some embodiments, upstream to the discharge nozzle, an air flow channel or chamber for conveying the substance released by the actuator to the discharge nozzle is arranged. The air flow channel may be, in particular, configured to support a turbulent air flow in the air flow channel. The turbulences in the air flow can facilitate entraining the particles released from the container and propel them towards the discharge nozzle of the device. Further, due to the turbulences in the air flow, the phase space occupied by the released particles can be increased such that a broad spatial distribution of the particle jet can be achieved. The broad spatial distribution of the particles may be particularly helpful to avoid local overdoses of the active ingredients at the exposed living tissues.

In some embodiments, the airflow channel is configured such that the air flow in the air flow channel can be created by the user by inhaling the air while keeping the nozzle of the device in a nostril or in the mouth. Such a device does not require any energy supply for providing the air flow.

The actuator can be configured to provide a pressurized air flow in the air flow channel. Release of pressurized air can, in particular, support turbulences which can help to entrain the substance particles located at an outlet of the container when the dosage valve is open.

In some embodiments, the device also comprises a chamber for propellant, and the actuator is configured to release the propellant such that the substance may be conveyed by droplets of propellant along the flow channel. As propellant, CFC (chlorofluorocarbon) and/or HFA (hydrofluoroalkane) may be used.

According to a further aspect, a substance for applying to a human or animal body for the treatment of lung tissue cells by inhalation. In particular the substance may be provided in the form of aerosol particles or powder particles. The substance comprises at least one active ingredient out of a group comprising in particular 2-DG. These active ingredients can serve as translation inhibitors for preventing or subduing viral replication in the human or animal cells and/or as agents for suppressing the growth and reproduction of the host cells attacked by viruses. Due to prevention of the viral replication, these active ingredients can also serve as a prophylaxis for preventing diseases or pathological developments caused by viral infections. The substance can be in particular applied to animals from a group comprising horses, swine, bovine animals as well as hen and/or other poultry.

In some embodiments, the particles may comprise a carrier material or matrix material carrying the active ingredient. The carrier material can be, in particular, provided in the form of a matrix in which the active ingredient resides. The carrier material can facilitate the handling and dispensing of the active agent.

The carrier material may comprise a liquid out of the group comprising water, alcohol, liquid glucose, or aqueous solution. The active ingredient or drug can be thus easily dispensed together with the liquid. The aqueous solution with 2% salt may serve as a preservation medium for the active agent until it is dispensed.

In some embodiments, the substance, in particular substance particles, comprise lactose and/or liposome. Lactose or liposome can serve as a carrier for the active ingredient, such that the particles can be easily delivered to the human body.

In some embodiments, the active agent is encapsulated in liposome, in order to achieve a long-lasting or retarded effect in the human body, thus increasing the duration of the desired effect. Particles may also comprise cholesterol, which stabilizes liposomes such that an even greater delay of the activation of the agent or active ingredient can be achieved.

The particles may comprise a mixture of different liposomes. In particular, a mixture of small liposomes, with an average size of less than 100 nm and large liposomes, with an average size of more than 150 nm. By providing different liposome sizes, a desired time profile of the active agent activity can be achieved.

The substance may be provided for use in the therapy of COVID-19 (Coronavirus Disease 2019), a respiratory disease caused by SARS-CoV-2 (severe acute respiratory syndrome coronavirus 2). The substance may, in particular, comprise a non-toxic concentration of the active ingredient, in particular 2-DG for preventing SARS-CoV-2 a replication in human lung tissue cells or in human nasal mucosa cells. The prevention of the replication of the SARS-CoV-2 in human lung tissue cells or in human nasal mucosa cells can help to avoid or mitigate the respiratory syndromes of the patients and, help not only in prophylaxis but also in the therapy of the COVID-19.

In particular, 2-DG molecules, due to the replacement of the 2-hydroxyl group by hydrogen, are characterized by high stability against metabolism. Due to the similarity with glucose molecules, 2-DG molecules penetrate the cells, undergo phosphorylation and resulting 2-DG-6-phosphate can remain for some time in the cell. However, in contrast to phosphorylated glucose, phosphorylated 2-DG-6-phophate does not further participate in glycolysis. Thus, 2-DG-6-phophate can remain in the cell, in particular, in the host cells attacked by a SARS-CoV-2 virus without undergoing glycolysis and, therefore, without producing energy necessary for cellular activities including biogenesis and reproduction of host cells. In other words, 2-DG takes the place of glucose, keeps the host cell busy, but does not produce energy, thus suppressing the growth and reproduction of the cells attacked by the SARS-CoV-2 virus.

Some parts of the embodiments have similar parts. The similar parts may have same names or similar part numbers. The description of one part applies by reference to another similar part, where appropriate, thereby reducing repetition of text without limiting the disclosure.

### BRIEF DESCRIPTION OF THE FIGURES

The application will be better understood and objects other than those set forth above will become apparent when consideration is given to the following detailed description thereof. Such description makes reference to the annexed drawings, wherein:

FIG. 1 Structural comparison of glucose and 2-deoxy-D-glucose (2-DG).

FIG. 2 Analysis of Spike protein glycosylation in human bronchial epithelial cells infected with SARS-CoV-2 in the presence and absence of 2-DG.

FIG. 3 Analysis of Spike protein glycosylation in normal renal epithelial cells infected with SARS-CoV-2 in the presence of 2-DG with dose-response relationship.

FIG. 4 Determination of IC50 values for 2-DG in human bronchial epithelial cells (short exposure).

FIG. 5 Determination of IC50 values for 2-DG in human bronchial epithelial cells (long exposure).

FIGS. 6a-6b Evaluation of antiviral activity of 2-DG on blocking the infection and replication of SARS-CoV-2 in primary bronchial epithelial cells.

FIGS. 7a-7b Evaluation of antiviral activity of 2-DG on blocking the infection and replication of SARS-CoV-2 in Vero E6 cell line.

FIGS. 8a-8c Evaluation of antiviral activity of 2-DG on blocking the multiplication of SARS-CoV-2 virus 8 hours after infection (post-treatment) in Vero E6 cell line.

FIGS. 9a-9b Analysis of Spike protein glycosylation in lysates obtained directly from normal renal epithelial cells infected with SARS-CoV-2 virus, in the presence and absence of 2-DG and with dose-response relationship.

FIG. 10 Uptake measurement of 2-DG in lung tissue lysates of mice treated with inhalation with 2-DG.

FIG. 11 Schematic drawing of a compound particle according to an exemplary embodiment. Diagram of a particle formed by spray drying a solution containing 2-DG. The black points are 2-DG contained in the particle structure.

FIG. 12 Size distribution chart of the particles containing 20% by weight of the 2-DG produced in example 10.

FIG. 13 Circularity chart of the particles containing 20% by weight of the 2-DG produced in example 10.

FIG. 14 Schematic drawing of a compound particle according to another exemplary embodiment. Diagram of a unilamellar liposome containing 300 mM of 2-DG solution within water space prepared in the examples 20-24.

FIG. 15 Schematic view of a device for inhaling a substance according to an exemplary embodiment.

FIG. 16 Flow chart of a method of dispensing a substance according to an embodiment.

### DETAILED DESCRIPTION

The following provides general remarks regarding the embodiments, examples, and aspects of the application.

In this text, the term viral disease caused by an enveloped virus comprising a spike protein refers to a viral disease which is caused by infection of a host with an enveloped virus comprising a spike protein as defined above. Spike proteins are visible structures in electron microscopes on the surface of such enveloped viruses. Exemplary enveloped viruses comprising a spike protein include Arenaviruses, Bunyaviruses, Coronaviruses (e.g. Sars-Cov-2 virus), Filoviruses, Flaviviruses, Hepadnaviruses, Herpesviruses, Orthomyxoviruses (e.g. Influenza virus), Paramyxoviruses, Poxviruses, Poxviruses, Retroviruses and Togaviruses.

In this text, the term glycosylation refers to the attachment of sugar moieties to proteins. It is a post-translational modification. Glycosylation is critical for a wide range of biological processes, including cell attachment to the extra-cellular matrix and protein-ligand interactions in the cell. This post-translational modification is characterized by various glycosidic linkages, including N-, O- and C-linked glycosylation, glypiation (GPI anchor attachment), and phosphoglycosylation.

In this text, the term viral envelope refers to the outer structure that encloses the nucleocapsids of some viruses.

In this text, the term spike protein refers to a glycoprotein that protrudes from the envelope of some viruses (such as e.g. a Coronavirus, Flaviviruses, Herpesviruses) and facilitates entry of the virion into a host cell by binding to a receptor on the surface of a host cell followed by fusion of the viral and host cell membranes.

In this text, the term effective tissue concentration of 2-DG relates to the amount of 2-DG that is available in an infected or unaffected tissue that is targeted for treatment by 2-DG such as e.g. a tissue of the lower respiratory tract, the heart or the liver.

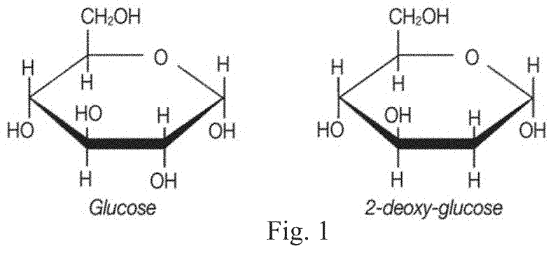

The application relates to 2-deoxyglucose (otherwise known as 2-deoxy-D-glucose or 2-Deoxy-D-xyl-hexose; hereinafter referred to as 2-DG) with the molecular formula C₆H₁₂O₅ for use in the treatment and prevention of viral infections caused by SARS-CoV-2, whose proteins require glycosylation to fold properly. 2-DG is a synthetic glucose analog where the C2 hydroxyl group is replaced with hydrogen (FIG. 1). 2-DG was provided by Sigma-Aldrich; catalog number D8375).

In the case of the SARS-CoV-2, the application found that that blocking or even hindering spike glycosylation is an effective way to inhibit infection because glycosylation of the spike protein is a requirement for infection.

The glycosylation dependent interaction between spike and ACE2 receptor has been described. Moreover, the SARS-CoV-2 virus multiplication in cells causes the rapid increase in energy demand of infected cells.

Blocking the glycosylation of the spike protein may have a positive effect also in the second stage of the disease, when viral remnants would not be able to play such a negative role due to the inactivation of a protein such as spike. For example, properly glycosylated and folded spike protein can penetrate into various cells and tissues, thereby leading to unfavorable effects. Blocking spike glycosylation therefore also plays a positive role in the second stage of the disease.

The application is based on the use of 2-DG in the treatment of Covid-19 and the prevention of SARS-CoV-2 virus infection of eukaryotic cells. The mechanism of action of 2-DG in this case is mainly based on the influence on the metabolism of infected cells, and in particular on blocking glycolysis and glycosylation of the SARS-CoV-2 virus spike protein. Spike glycosylation plays a fundamental role in infecting cells because the interaction between spike and its major ACE-2 receptor depends on glycosylation.

It is shown herein that 2-DG by preventing the spread of SARS-CoV-2 infection—by preventing virus multiplication in eukaryotic cells—has both a preventive effect in blocking the infection as observed when first exposing cells to 2-DG and subsequently to SARS-CoV-2, as well as a therapeutic effect as observed when first exposing cells to SARS-CoV-2 and subsequently to 2-DG or when exposing cells simultaneously to both. It is also shown herein that 2-DG completely inhibits the standard/regular glycosylation of the spike protein that is observed in the absence of 2-DG. Blocking these processes are effective at concentrations that are much lower than concentrations which cause toxic effects in normal cells.

To evaluate the toxicity of 2-DG to normal cells as well as to assess the inhibition of viral multiplication the human bronchial epithelial cells (HBEpiC) line and Vero6 cell line were obtained. 2-DG was supplied by Sigma-Aldrich (catalog number D8375).

In this aspect, the human bronchial epithelial cells (hereinafter referred to as HBEpiC) and primary human bronchial epithelial cells (hereinafter referred to as PBEC) are the same cells but provided from different vendors (from Innoprot (Cat no. P10557) and Promocell (C-12640), respectively). Thus, the phrases HBEpiC and PBEC are used interchangeably within text.

Thus, 2-DG exhibits many advantages making it a promising pharmaceutically active ingredient for use in a medical method to prevent and/or to treat a viral disease, in particular Covid-19, including advantages such as:
2-DG is non-toxic or minimally toxic or harmless against normal (uninfected) cells at concentrations which generally exhibit a therapeutic effect on infected cells;
2-DG exhibits only a slight toxicity compared to other drugs used against SARS-CoV-2;
2-DG reduces SARS-CoV-2 virus multiplication;
2-DG reduces multiplication of SARS-CoV-2 virus in cells infected with this virus before exposure to 2-DG;
2-DG reduces or blocks glycosylation of viral proteins, especially spike proteins;
2-DG reduces or blocks glycolysis in eukaryotic cells infected by SARS-CoV-2.

Liposomes are among the best drug delivery systems. They are most often non-toxic, biocompatible and can be made up of the same components as the cells of the human body. Liposomes can be used intravenously, intraperitoneally, orally and pulmonally.

There are some limitations to the use of liposomes, however. In the case of long storage of liposome preparations, leakage of the encapsulated substances, crystallization or their gradual hydrolysis in water is observed. To avoid these problems, it is possible to use so-called proliposomes. These can be both lyophilized liposomes as well as lyophilized lipids (or organic solutions of lipids with API) which form liposomes in an aqueous solution due to the natural features of phospholipids related to their structure.

A further aspect of the application relates to a proliposome preparation containing the active substance 2-deoxyglucose (2-DG).

As described above and below, 2-deoxyglucose (otherwise known as 2-deoxy-D-glucose or 2-Deoxy-D-xyl-hexose; herein referred to as 2-DG), with the molecular formula C6H12O5, is a synthetic glucose analog where the C2 hydroxyl group is replaced with hydrogen. 2-DG was provided by Sigma-Aldrich; catalog number D8375).

2-DG is a simple reducing sugar which is very difficult to maintain in the form of very small particles because of their relatively high hygroscopicity and low glass transition temperature resulting of stacking together sticky particles.

One of the solutions to the problem of both 2-DG spray drying of glucose and obtaining delayed release of this substance in the lungs is the possibility of using proliposomes.

### The Preparation of Proliposomes

In the case of 2-DG solution spray drying mixed with solution of other excipients and phospholipids such as naturally occurring dipalmitoyl phosphatidylcholine (DPPC) in the lungs, it is possible to obtain particles wherein 2-DG and excipients are mixed with phospholipids to form particles of small size.

The hydration of such particles causes the organization of phospholipids into the lipid bilayer and the partial entrapment of the substances that formed the particle in the water space of the liposomes. Leucine is an excipient that significantly improves the aerological properties of the particles obtained by spray drying. In the case of drying from an organic-water solution, its diffusion to the surface of the particles is observed. This allows for the surrounding of a more hydrophilic 2-DG inside the particles and for obtaining another phospholipid layer above or below the leucine layer, depending on the drying method and the solvents used.

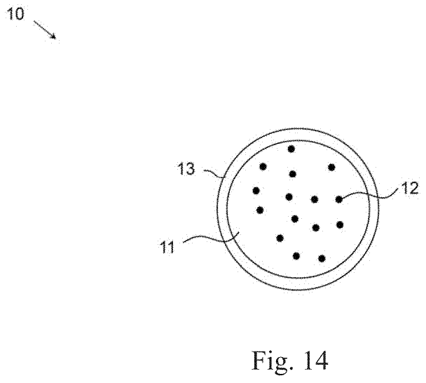

After hydration, the obtained particles form liposomes which partially encapsulate 2-DG (FIG. 14). 2-DG encapsulation efficiency depends on the shell composition and phospholipid content and mixture of solvents used. In some embodiments, the active agent is encapsulated in proliposomes in which the amount of active ingredient ranges from 1 to 80%. Each particle formed as a result of spray drying gives one liposome, the size of which depends on the amount of phospholipid and the fluidity of the lipid bilayer of the liposomes. Stiffer membranes produce smaller liposomes and more fluid membranes produce larger liposomes. This effect is due to difference in osmotic pressure after the substance is encapsulated which then causes the causes the liposomes to swell. Thereby, liposomes with more fluid membranes increase in size, whereas liposomes with stiff membranes break apart.

In general to produce proliposomes by spray-drying process excipients such as mannitol, trehalose, amino acids (glycine, leucine) and phospholipids forming the lipid bilayer) can be used. Preferably, the formulation of proliposomes contains mannitol in the amount of 0 to 60% by weight of the preparation, glycine or leucine amino acid in the amount of 0 to 80% by weight of the preparation and trehalose in the amount of 0 to 60% by weight of the preparation. Additionally, cholesterol and negatively or positively charged phospholipids can be used, using them in small amounts as liposome bilayer stabilizers. Examples of negatively charged phospholipid species include natural phosphatidylglycerols, dimyristoyl phosphatidylglycerol, dipalmitoylphosphatidylglycerol, and other phospholipids. In some embodiments, the nicotinic acid amide was used in proliposome formulation to facilitate phospholipids dissolving. Molten nicotinic acid amide dissolves phospholipids and many poorly soluble substances.

The ratio of phospholipids to active substance and excipients determines the amount of substances in the internal water space of liposomes. More often the more phospholipids, the higher the entrapment efficiency of the active ingredient and the proliposome-forming excipient. But this parameter is also related to the nature of the excipients and other factors.

Preferably a suitable ratio between the phospholipids and the excipient and active substance should be from 1 to 10 to 1 to 1. The parameter determining the rate of release of the active substance from liposomes is the phase transition temperature of the membrane of the liposomes used in the formulation. In the case of liposomes composed of DPPC alone, the release rate of the active substance can be very slow. By changing the composition of the lipid bilayer by using increasing amounts of DMPC, it is possible to obtain a bilayer with ever greater permeability to active substances, and thus it is possible to control the leakage of this substance over a wide time range. DPPC is a lipid whose phase transition temperature is around 41.5° C. Below temperature, this lipid does not form a lipid bilayer, therefore an addition (10 to 30 molar percent) of another phospholipid (DMPC) with lower phase transition temperature is important. Preferably a suitable ratio between DPPC and DMPC should be from 1 to 10 to 10 to 1. The use of a formulation containing DPPC and DMPC in such a weight ratio that causes a phase transition at the temperature of the human body causes a rapid release of the entire content of liposomes within several minutes. By increasing the proportion of DMPC to DPPC by a few percent by weight can extend the release time of the substance to several hours. It is therefore possible to consciously control the release time of the encapsulated 2-DG by varying the ratio of DMPC to DPPC and by changing the lamellarity of liposomes.

Another approach to proliposomes production is the use of API and lipids (phospholipids or phospholipid and sterols) solution dissolved in the water/organic cosolvents system being able to dissolve both API and lipids. This solution when nebulized forms liposomes upon contact with water. The API is the partially encapsulated and the encapsulation efficiency depends on API concentration, API lipids ratio and its concentration within water/organic cosolvent system. The pharmaceutically approved solvents, miscible with water which dissolves lipids are propylene glycol, glycerol and ethyl alcohol and can be used alone or in desired combination. The addition of water do not produce lipids precipitation until certain water concentration is achieved. In many cases the water soluble substance can be dissolved in such a solution to form API lipid solution. This solution if mixed with water forms multilamellar, oligolamellar or unilamellar liposomes, depending of the lipids concentration, organic excipients type or temperature. The solution can be nebulized by commercially available systems to form very fine solution droplets containing 2DG phospholipids or phospholipids and sterols which form different types of liposomes with varying 2DG encapsulation efficiency.

Alternatively, 2-DG proliposomes can be prepared by dehydration-rehydration method followed by extrusion method in order to achieve large unilamellar liposomes. Non encapsulated 2-DG in liposomes can remain in suspension and does not need to be separated. The encapsulation efficiency varies from 25-50% depending the method used, liposomes composition and size. The resulting suspension is then mixed with water/ethanol solution of excipients such like leucine and trehalose and spray-dried. Preferably, formulation of proliposomes contains trehalose in the amount of 0 to 60% by weight of the preparation. Resulting proliposomes particles have the size of 1-3 μm depending of the final mixture composition, concentration and spray-drying conditions. The non-encapsulated 2-DG exerts its local activity immediately while the encapsulated is slowly released and constitutes a depot that gradually replenishes the metabolism of free 2-DG.

2-DG can be also used for intravenous administration. In order to extend the circulation time a proper type of liposomes must be used. Liposomes of a size close to 100 nm with 2-DG may be encapsulated within the internal space of liposomes by one of the available passive methods of drug encapsulation. In some embodiments, the liposomes used for intravenous delivery have sizes from 30 nm to 200 nm. 2-DG can also be also used for pulmonary application. In some embodiments, the liposomes used to pulmonary delivery have sizes ranging from 50 nm to 1 μm.

The phospholipids which can be used include: hydrogenated soya or egg PC, DPPC, DSPC, DPPG and DSPG. Also natural egg sphingomyelin can be used as a base for liposomal composition.

The liposomes may contain cholesterol. Most often a preferable range of 30 to 50 molar percent in the relation to other lipids is be applied. Liposomal composition can be further enriched in polymer modified lipids in order to extend their circulation time. As a polymer polyethylene glycol, polyvinyl alcohol or polyglycerol may be used. The 2-DG will be encapsulated by one of existing method such like dehydration-rehydration method, polyol dilution method or the passive equilibration method which utilize the 30% ethanol in liposomes suspension. The size of the liposomes may be controlled by liposomes extrusion, high pressure homogenization or a similar technique. Non encapsulated 2-DG may be removed by size exclusion chromatography or dialysis.

### Figure Legends

FIG. 1. Structural comparison of glucose and 2-deoxy-D-glucose.

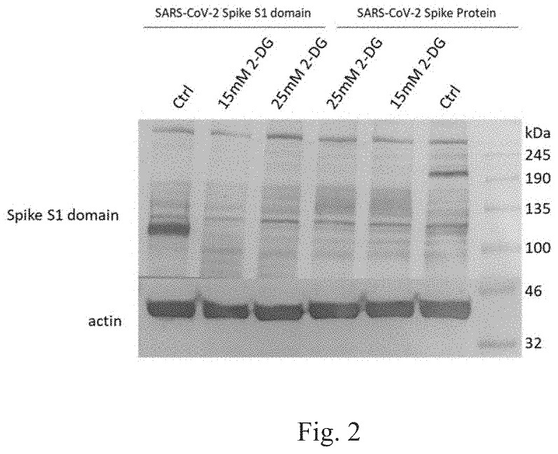

FIG. 2. Analysis of Spike protein glycosylation in human bronchial epithelial cells (HBEpiC) showing that 2-DG inhibits glycosylation of spike protein from SARS-CoV-2 virus. “Ctrl” means that cells were not exposed to 2-DG. Each monomer of Spike protein is estimated to be 180 kDa in size.

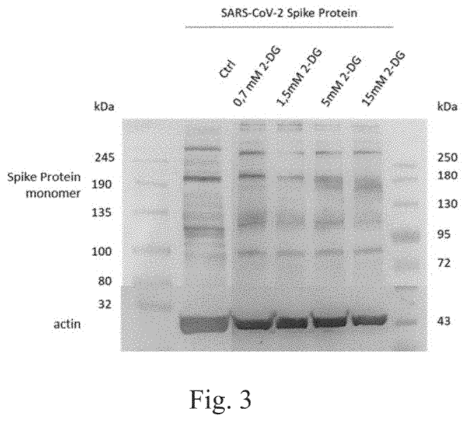

FIG. 3. Analysis of Spike protein glycosylation in normal renal epithelial cells (Vero-E6) showing that 2-DG inhibits glycosylation of spike protein from SARS-CoV-2 virus. “Ctrl” means that cells were not exposed to 2-DG. The dose-response relationship for concentrations from 0.7 mM to 15 mM is clearly visible. Each monomer of Spike protein is estimated to be 180 kDa in size.

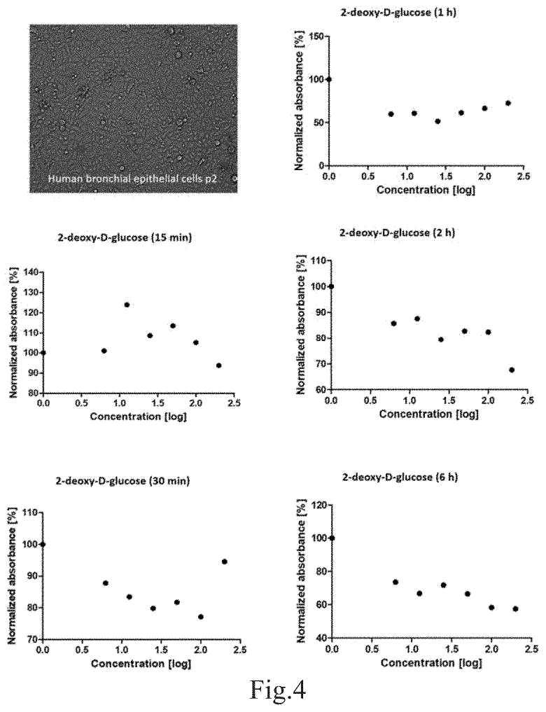

FIG. 4. Determination of IC50 values for 2-deoxy-D-glucose in human bronchial epithelial cells (short exposure) revealed lack of cytotoxic effects on epithelial cells after 15 minutes to 6 hours exposure to 2-DG. An IC50 value cannot be determined due to lack of toxicity.

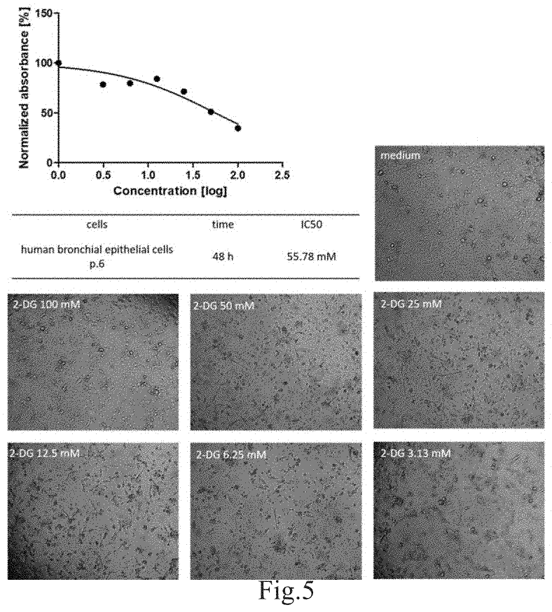

FIG. 5. Determination of IC50 values for 2-deoxy-D-glucose in human bronchial epithelial cells (long exposure) revealed very high IC50 value after exposing human epithelial cells to 2-DG.

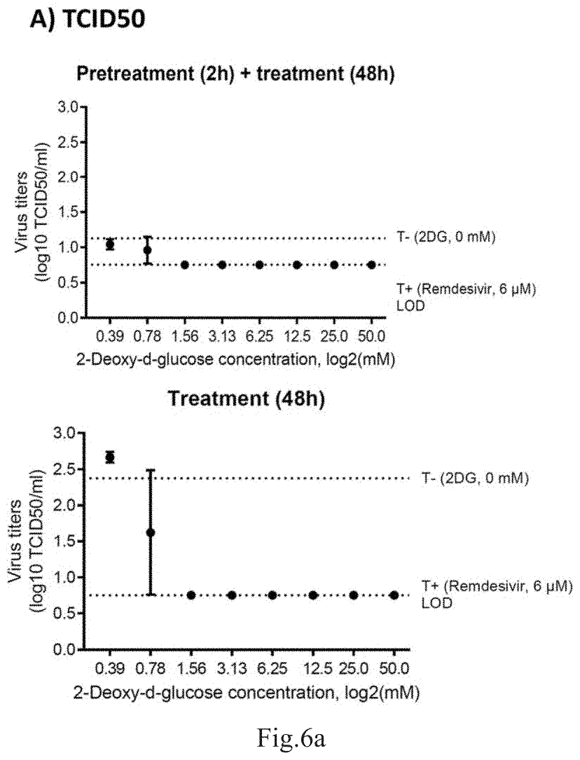

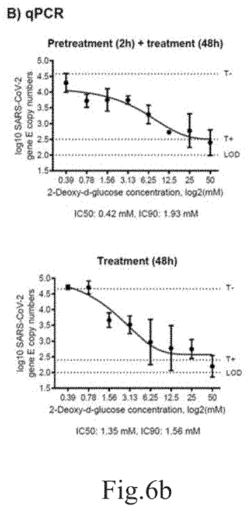

FIGS. 6a-6b. Evaluation of antiviral activity of 2-DG on blocking the infection and replication of SARS-CoV-2 in primary bronchial epithelial cells (PBEC). 2-DG induces a dose dependent decrease in SARS-CoV-2 virus production on primary human bronchial epithelial cells, with effect equivalent to the positive control remdesivir above 0.78 mM. Effect of increase concentrations of 2-deoxy-D-glucose on SARS-CoV-2 replication in primary human bronchial epithelial cells (HBEpiC). Limit of detection (LOD) and viral titers without compound (T−) or with remdesivir (T+, 6 μM—3 times the EC90) are indicated by dotted lines. Tested compound was used at 0.39, 0.78, 1.56, 3.13, 6.25, 12.5, 25 and 50 mM. A) Viral titers were determined by TCID50 method on Vero-E6 cells and calculated by the Spearman and Karber algorithm. B) Copy numbers of gene E of SARS-CoV-2 were determined by TaqMan One Step RT-qPCR with E_Sarbeco primers and probe (Charite, Corman et al Eurosurveillance; PMID:31992387) and following instructions of the Qiagen QuantiNova Probe RT-PCR Kit. IC50 and IC90, calculated from nonlinear regressions, are indicated below. TCID50 value=infectious virus titer.

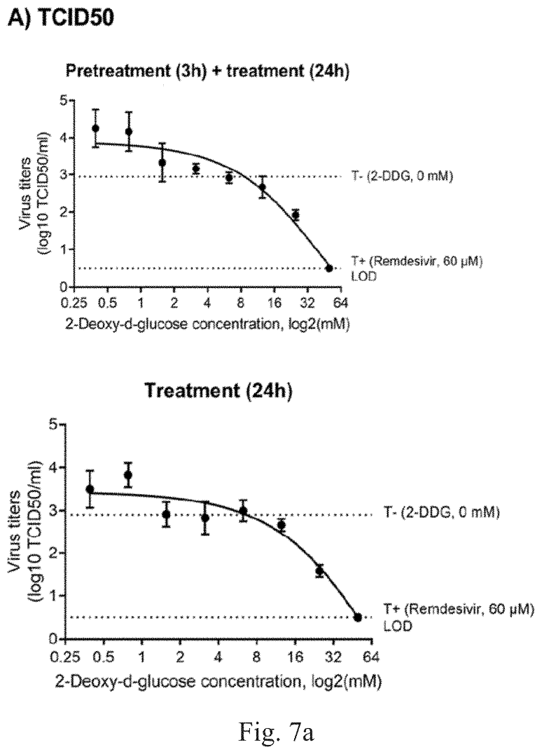

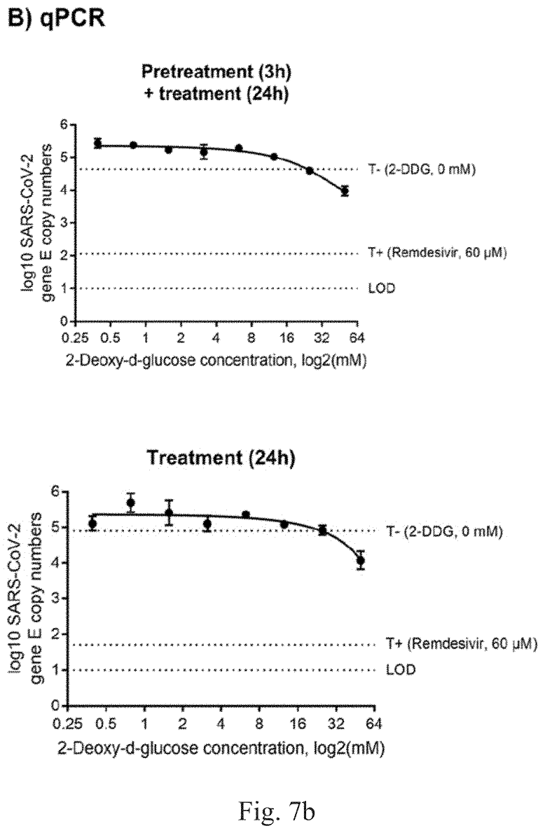

FIGS. 7a-7b. Evaluation of antiviral activity of 2-DG on blocking the infection and replication of SARS-CoV-2 in Vero E6 cell line. 2-DG induces a strong decrease in the production of infectious SARS-CoV-2 particles. Effect is impressive on TCID50 calculation but less pronounced on qPCR experiments. Effect of increase concentrations of 2-deoxy-D-glucose on SARS-CoV-2 replication in Vero E6 cells. Limit of detection (LOD) and viral titers without compound (T−) or with remdesivir (T+, 6 μM—3 times the EC50) are indicated by dotted lines. Tested compound was used at 0.39, 0.78, 1.56, 3.13, 6.25, 12.5, 25 and 50 mM. A) Viral titers were determined by TCID50 method on Vero-E6 cells and calculated by the Spearman and Karber algorithm. B) Copy numbers of gene E of SARS-CoV-2 were determined by TaqMan One Step RT-qPCR with E_Sarbeco primers and probe (Charite, Corman et al Eurosurveillance; PMID:31992387) and following instructions of the Qiagen QuantiNova Probe RT-PCR Kit. IC50 and IC90, calculated from nonlinear regressions, are indicated below.

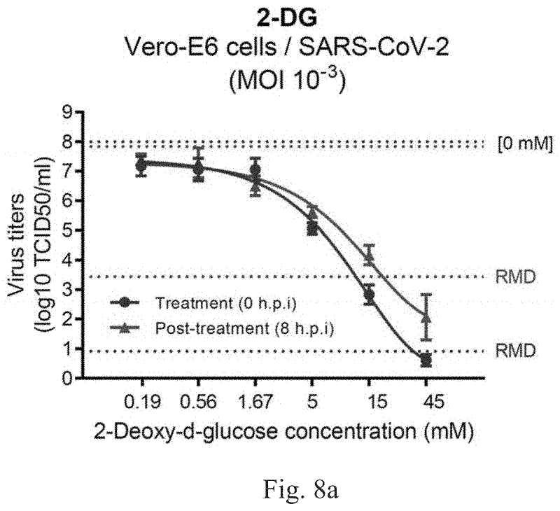

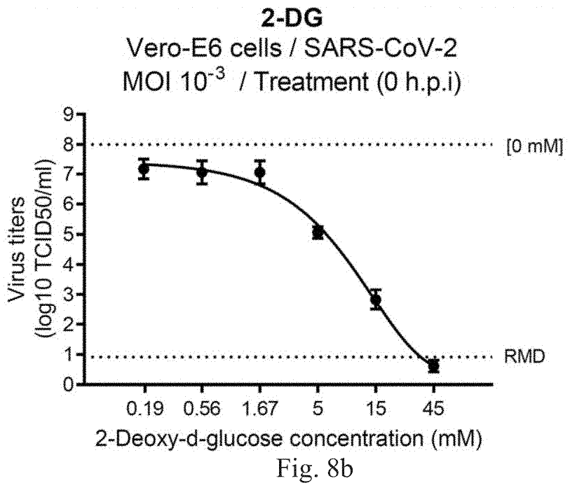

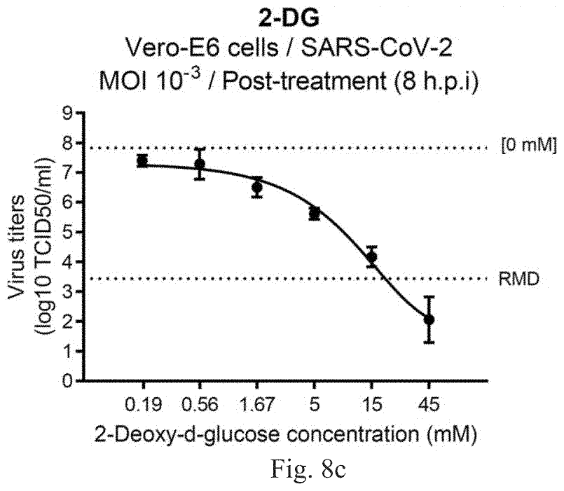

FIGS. 8a-8c. Effect of increase concentrations of 2-Deoxy-D-glucose on SARS-COV-2 replication in Vero E6 cells. Viral titers without compound (“[0 μM]”) or with 6 μM of remdesivir (“RMD”) are indicated by dotted lines. Tested compound was used at 0.19, 0.56, 1.67, 5, 15, and 45 mM. 2-DG was added at 0 h.p.i for the “treatment” group and at 8 h.p.i for the “post-treatment” group. Viral titers were determined by the TCID50 method on Vero-E6 cells and calculated by the Spearman & Karber algorithm. Non-linear curve fitting are indicated by full lines for A) “treatment” (blue) and “post-treatment” (red) groups, B) “treatment” group only and C) “post-treatment” group only.

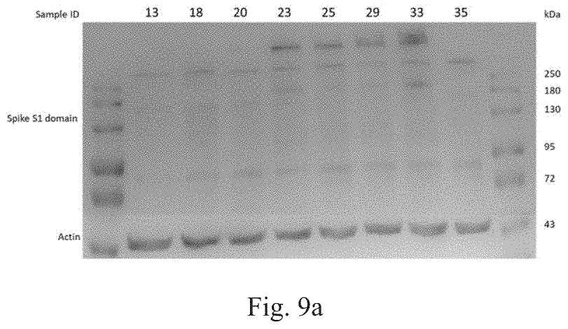

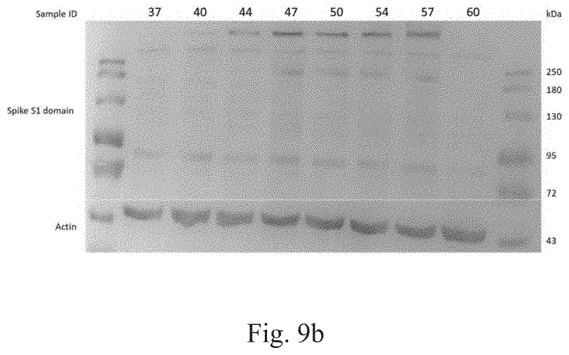

FIGS. 9a-9b. Analysis of Spike protein glycosylation in Vero-E6 cells showing that 2-DG inhibits glycosylation of Spike protein of SARS-CoV-2 virus isolated directly from cells infected with this virus. Line “33” and line “57” represent lysates from cells that were not exposed to 2-DG.

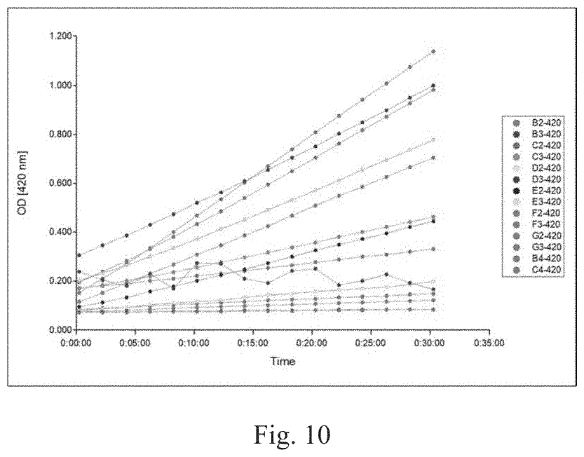

FIG. 10. Uptake measurement of 2-DG in lung tissue lysates of mice treated with inhalation with 2-DG showing that 2-DG was accumulated in the lungs of mice in this animal model study. Temporal changes of OD for all the samples included in the analysis are visible.

### EXAMPLES

### Example 1. Analysis of Spike Protein Glycosylation in Human Bronchial Epithelial Cells

Human Bronchial Epithelial Cells (HBEpiC) were obtained from Innoprot (Cat no. P10557, batch no. 7475) and cultured in Bronchial Epithelial Cell Medium (Innoprot, Cat no. P60151) supplemented with 0.2% gentamicin (Gibco, Life Technologies, USA, Cat. no. 15710-049) under standard cell culture conditions (5% CO₂, 16% O₂, 37° C.). HBEpiC at passage 2, were plated in Bronchial Epithelial Cell Medium at 297.0 thousand cells per well of a 6-well plate and left for 48 hours in an incubator. The plates were coated with collagen (Collagen I-cell surface coating kit, Innoprot, Cat no. P8188) prior to cell seeding. The cells were transduced with lentiviral vectors encoding the SARS-CoV-2 Spike Protein or Spike S1 domain. After 24 h post transduction, 25 mM or 15 mM of 2-deoxy-D-glucose (Sigma-Aldrich; Cat no. D8375) was added to the cells for next 24 h. The cells were lysed for 30 min at 4° C. in cell lysis buffer (50 mM Tris-HCl pH 7.5, 1 mM EDTA, 1 mM EGTA, 1 mM Sodium Orthovanadate, 10 μM β-glycerophosphate, 5 μM Sodium Pyrophosphate and 0.5% Triton X-100) freshly supplemented with Proteases and Phosphatases Inhibitor Cocktail. Lysates were clarified at 7,000×g for 6 min at 4° C. The samples were mixed with Laemmli with β-mercaptoethanol, heated at 98° C. for 3 minutes and 25 μg (as determined by BCA assay (Thermo Scientific, Cat. no. 23225)) of the proteins were applied to the gel for Western Blot analysis. Bands were visualized using Opti-4CN Substrate Kit (Bio-Rad, Cat. no. 1708235).

Antibodies that were used in this study included:
anti-SARS-CoV-2 Spike Glycoprotein S1 antibody (Abcam, Cat. no. ab275759), dilution 1:500;
anti-Actin Antibody, clone C4 (Sigma-Aldrich, Cat. no. MAB1501), dilution 1:4000;
1:4000;
anti-rabbit IgG-HRP (Santa Cruz Biotechnology, Cat. no. sc-2357), dilution
anti-mouse IgG-HRP (Santa Cruz Biotechnology, Cat. no. sc-516102), dilution 1:4000.

Results and conclusions: The experiment shows that 2-DG inhibits glycosylation of spike protein from SARS-CoV-2 virus (FIG. 2). The effect of dose and timing of dose (the dose-response relationship as well as the time-response relationship) of 2-DG on blocking of spike glycosylation is observed in the concentration range of 10 μM (0.01 mM) to 10 mM. In FIG. 2 at a concentration of 15 mM total inhibition was observed.

### Example 2. Analysis of the Glycosylation of Spike Protein in Normal Renal Epithelial Cells

Renal epithelial cells (VERO, clone E6) were obtained from ATCC (Cat no. CRL-1586, batch no. 70034994) and cultured in Eagle's Minimum Essential Medium (ATCC, Cat no. 30-2003) supplemented with 0.2% gentamicin (Gibco, Life Technologies, USA, Cat. no. 15710-049), 1% Penicillin with Streptomycin (Biowest, Cat. no. L0022-100) and 10% fetal bovine serum (Biowest, Cat. no. S181H-500) under standard cell culture conditions (5% CO2, 16% O2, 37° C.). Vero E6 cells at passage 4, were plated in EMEM at 297.0 thousand cells per well of a 6-well plate and left for 24 hours in an incubator. The cells were transduced with lentiviral vectors encoding the SARS-CoV-2 Spike Protein. After 24 h post transduction, 0.7 mM, 1.5 mM, 5 mM and 15 mM of 2-deoxy-D-glucose (Sigma-Aldrich; Cat no. D8375) was added to the cells for next 24 h. The cells after incubation with 2-deoxy-D-glucose (or without this compound as in control group) were lysed for 30 min at 4° C. in cell lysis buffer (50 mM Tris-HCl pH 7.5, 1 mM EDTA, 1 mM EGTA, 1 mM Sodium Orthovanadate, 10 μM β-glycerophosphate, 5 μM Sodium Pyrophosphate and 0.5% Triton X-100) freshly supplemented with Proteases and Phosphatases Inhibitor Cocktail. Lysates were clarified at 7,000×g for 6 min at 4° C. The samples were mixed with Laemmli with β-mercaptoethanol, heated at 98° C. for 3 minutes and 25 μg (as determined by BCA assay (Thermo Scientific, Cat. no. 23225)) of the proteins were applied to the gel for Western Blot analysis. Bands were visualized using Opti-4CN Substrate Kit (Bio-Rad, Cat. no. 1708235).

Antibodies used in the study:
anti-SARS-CoV-2 Spike Glycoprotein S1 antibody (Abcam, Cat. no. ab275759), dilution 1:500
anti-Actin antibody, clone C4 (Sigma-Aldrich, Cat. no. MAB1501), dilution 1:4000
anti-rabbit IgG-HRP (Santa Cruz Biotechnology, Cat. no. sc-2357), dilution 1:4000
anti-mouse IgG-HRP (Santa Cruz Biotechnology, Cat. no. sc-516102), dilution 1:4000

Results and conclusions: The experiment shows that 2-DG inhibits glycosylation of spike protein from SARS-CoV-2 virus (FIG. 3). The effect of dose and timing of dose (the dose-response relationship as well as the time-response relationship) of 2-DG on blocking of spike glycosylation is observed in the concentration range of 10 μM (0.01 mM) to 10 mM. In FIG. 3 the dose-response relationship is clearly visible (selected concentrations for this blot ranges from 0.7 mM to 15 mM).

### Example 3. Determination of IC50 Values for 2-Deoxy-D-Glucose in Human Bronchial Epithelial Cells (Short Exposure)

Human Bronchial Epithelial Cells (HBEpiC) were obtained from Innoprot (Cat no. P10557, batch no. 7475) and cultured in Bronchial Epithelial Cell Medium (Innoprot, Cat no. P60151) supplemented with 0.2% gentamicin (Gibco, Life Technologies, USA, Cat. no. 15710-049) under standard cell culture conditions (5% CO₂, 16% O₂, 37° C.). HBEpiC at passage 2, were plated in Bronchial Epithelial Cell Medium at 10.0 thousand cells per well of a 96-well plate and left for 48 hours in an incubator. The plates were coated with collagen (Collagen I-cell surface coating kit, Innoprot, Cat no. P8188) prior to cell seeding. 2-deoxy-D-glucose (Sigma-Aldrich; Cat no. D8375) was added to the cells for 15 min, 30 min, 1 h, 2 h and 6 h.

Cells were exposed to the compound at the following concentrations of 2-deoxy-D-glucose in medium as a solvent: 200 mM; 100 mM; 50 mM; 25 mM; 12.5 mM; 6.25 mM.

In order to assess cell viability, medium containing 2-deoxy-D-glucose was removed, the fresh medium was added to the cells and CellTiter 96® AQueous Solution Assay (Promega) was used in accordance with the manufacturer's instructions, 20 μl of reagent was added per 100 μl of cell culture medium and cells were incubated at 5% CO2, 16% O2, 37° C. Absorbance was measured at 490 nm. The results obtained 1 hour after addition of the CellTiter reagent were analyzed in GraphPadPrism 5.01. Data normalization and a nonlinear regression model were applied in order to determine the IC50.

Results and conclusions: The experiment shows lack of cytotoxic effects on epithelial cells after 15 minutes to 6 hours exposure to 2-DG. 2-deoxy-D-glucose at a concentration of even 200 mM within this range of incubation times did not cause cell death in a sufficient amount allowing for a IC50 calculation (FIG. 4).

### Example 4. Determination of IC50 Values for 2-Deoxy-D-Glucose in Human Bronchial Epithelial Cells (Long Exposure)

Human Bronchial Epithelial Cells (HBEpiC) were obtained from Innoprot (Cat no. P10557, batch no. 7475) and cultured in Bronchial Epithelial Cell Medium (Innoprot, Cat no. P60151) supplemented with 0.2% gentamicin (Gibco, Life Technologies, USA, Cat. no. 15710-049) under standard cell culture conditions (5% CO₂, 16% O₂, 37° C.). HBEpiC at passage 6, were plated in Bronchial Epithelial Cell Medium at 9.0 thousand cells per well of a 96-well plate and left for 48 hours in an incubator. The plates were coated with collagen (Collagen I-cell surface coating kit, Innoprot, Cat no. P8188) prior to cell seeding. 2-deoxy-D-glucose (Sigma-Aldrich; Cat no. D8375) was added to the cells for 48 h.

Cells were exposed to compounds at the following concentrations of 2-deoxy-D-glucose in medium as a solvent: 100 mM; 50 mM; 25 mM; 12.5 mM; 6.25 mM; 3.13 mM.

In order to asses cell viability, CellTiter 96® AQueous Solution Assay (Promega) was used in accordance with the manufacturer's instructions, 20 μl of reagent was added per 100 μl of cell culture medium and cells were incubated at 5% CO2, 16% O2, 37° C. Absorbance was measured at 490 nm. The results obtained 4 hours after addition of the CellTiter reagent were analyzed in GraphPadPrism 5.01. Data normalization and a nonlinear regression model were applied in order to determine the IC50.

Results and conclusions: The experiment shows a very high IC50 value after exposing human epithelial cells to 2-DG (FIG. 5).

### Example 5. Evaluation of 2-DG Antiviral Activity in Primary Bronchial Epithelial Cells (PBEC) Infected by SARS-CoV-2 Virus

Work Plan:

1. Reception and amplification of PBEC cells.

2. Seeding of PBEC and calibration of the infection with SARS-CoV-2.

3. If calibration is successful, treatment with compound of interest in triplicate.

4. Infection with SARS-CoV-2 at one MOI (multiplicity of infection).

5. Recovery of viral particles in the supernatant after incubation and quantification using TCID50 (tissue culture infectious dose 50%) and RT-qPCR on Vero E6 cells. Data analysis.

Calibration Protocol:

Day 1. Human PBEC cells from Promocell (C-12640) in passage 3 were seeded in 48 wells plate. Growth conditions: Airway epithelial cell growth medium (Promocell C-21060).

Day 2. Infection with SARS-CoV-2 (1 h with either 1, 2 or 3×PBS wash) at MOI 10⁻¹.

Day 3. Recovery of virus particle and measurement of the production by RT-qPCR on triplicate pool.

Screening Protocol:

Day 1. PBEC human cells from Promocell (C-12640) were seeding in 48 wells plate in growth conditions included Airway epithelial cell growth medium (Promocell C-21060).

Day 2. Pre-treatment of PBEC cells in 48 wells plate with 2-DG prior to SARS-CoV-2 infection (2 h), and treatment with 2-DG for 48 h. SARS-CoV-2 was added at MOI 10-1 (MOI 0.1) and removed after 2 hours. Then, the cell culture was washed with PBS (3×0.5 mL) and 300 μL of medium with 2-DG from 50 mM to 0.39 mM, or medium with or without remdesivir for control, were added.

Day 4. Viral titer was assessed by the TCID50 (Median Tissue Culture Infectious Dose) method on Vero-E6 cells and calculated by the Spearman & Karber algorithm.

Infection process was conducted according to below presented scheme:

T=−2 h or t=0 Treatment with compound from 50 mM to 0.39 mM

T=0 h Infection with SARS-CoV-2 at MOI 10⁻¹ (MOI 0.1) with 2-DG 50 mM to 0.39 mM

T=2 h Virus removal and PBS wash (3×0.5 mL). Add 300 μL medium with 2-DG from 50 mM to 0.39 mM

[48 h incubation]

T=50 h Recover 150 μL of supernatant and TCID50 processing
RT-qPCR targeting SARS-CoV-2 E gene

[4 day incubation]

Endpoint TCID50 reading and calculation.

Results and conclusions: It was demonstrated that 2-DG influences on SARS-CoV-2 propagation in human epithelial cells (FIGS. 6a-6b). In the conditions with 2 h Pretreatment (according to above listed schedule) 2-DG induces a decrease in the production of infectious SARS-CoV-2 particles starting at 0.78 mM. In Treatment conditions only SARS-CoV-2 virus production was stronger (around 2.5 log 10 (TCID50)). In these conditions 2-DG induces a massive decrease in the production of infectious SARS-CoV-2 particles starting at 0.78 mM. At conditions above 0.78 mM, 2-DG effect is equivalent to the positive control remdesivir.

To conclude, 2-DG induces a dose dependent decrease in SARS-CoV-2 virus production on primary human bronchial epithelial cells, with effect equivalent to the positive control remdesivir above 0.78 mM. No toxicity of 2-DG was observed even during the 48 h incubation. 2-DG concentration required to inhibit SARS-CoV-2 replication in human epithelial cells was at least 50 times lower than IC50 concentration.

### Example 6. Evaluation of Antiviral Activity of 2-DG on Blocking the Infection and Replication of SARS-CoV-2 in Vero E6 Cell Line

Work Plan:

Seeding of cells and treatment with compound of interest in triplicate.

Infection with SARS-CoV-2 at one MOI (multiplicity of infection).

Recovery of viral particles in the supernatant after incubation and quantification using TCID50 (tissue culture infectious dose 50%) and RT-qPCR. Data analysis.

Screening Protocol:

Day 1: Vero E6 cells in passage 41 were seeding in 96 wells plate in growth conditions including medium DMEM with high glucose (Dutscher L0104-500, lot MS008A).

Day 2: 2-DG was diluted at 1M in medium and added to a final concentration of 50 mM, than diluted/2 in triplicate until 0.39 mM.

In parallel: toxicity assessment on Vero cells treated with 2-DG at the same concentrations, fixed at 24 h post treatment and tested with a cytotoxicity algorithm (number of cells, nuclear morphology) in two media (DMEM high glucose and F12 low glucose).

Infection process was conducted according to below presented scheme:

T=−3 h or t=0 Treatment with compound from 50 mM to 0.39 mM

T=0 h Infection with SARS-CoV-2 at MOI 10⁻³ (MOI 0.001) with 2-DG 50 mM to 0.39 mM

T=1 h Virus removal and PBS wash (2×1 mL). Add 1 mL medium with 2-DG from 50 mM to 0.39 mM

[24 h incubation]

T=25 h Recover 500 μL of supernatant and TCID50 processing
RT-qPCR targeting SARS-CoV-2 E gene

[4 day incubation]

Endpoint TCID50 reading and calculation.

Results and conclusions: 2-DG induces a strong decrease in the production of infectious SARS-CoV-2 particles in Vero E6 cells (FIGS. 7a-7b). Effect is impressive on TCID50 calculation but less pronounced on qPCR experiments. Effect of increase concentrations of 2-deoxy-D-glucose on SARS-CoV-2 replication in Vero E6 cells is clearly visible.

### Example 7. Evaluation of Antiviral Activity of 2-DG on Blocking the Multiplication of SARS-CoV-2 in Vero E6 Cell Line 8 Hours after Infection (Post-Treatment) in Vero E6 Cell Line

Work Plan:

1. Seeding of cells and treatment with compound of interest in triplicate.

2. Infection with SARS-CoV-2 at one MOI (multiplicity of infection).

3. Recovery of viral particles in the supernatant after incubation and quantification using TCID50 (tissue culture infectious dose 50%). Data analysis.

Screening Protocol:

Day 1: Vero E6 cells were seeding in 96 wells plate in growth conditions including medium DMEM with high glucose (Dutscher L0104-500, lot MS008A).

Day 2: 2-DG was diluted at 1M in medium and added to a final concentration of 45 mM, then diluted/3 in triplicate until 0.19 mM.

In parallel: toxicity assessment on Vero cells treated with 2-DG at the same concentrations, fixed at 24 h post treatment and tested with a cytotoxicity algorithm (number of cells, nuclear morphology) in two media (DMEM high glucose and F12 low glucose).

Infection with SARS-CoV-2 at MOI equal 10-3 was conducted at T=0 h and 2-DG was added at 0 h.p.i for the “treatment” group and at 8 h.p.i for the “post-treatment” group.

Results and conclusions: 2-DG induces a strong decrease in the production of infectious SARS-CoV-2 particles in Vero E6 cells when added 8 hours after cells infection (FIGS. 8a-8c). Effect is impressive on TCID50 calculation. Effect of increase concentrations of 2-deoxy-D-glucose on SARS-CoV-2 replication in Vero E6 cells is clearly visible. Results showed that 2-DG can be used not only in preventing but also in treatment of COVID-19 by suppressing viral replication.

### Example 8. Analysis of the Glycosylation of Spike Protein in Renal Epithelial Cells

The analysis was performed on the samples (see table below) obtained within experiment described in example 6.

| | | | | Number of | | Treatment/ | 2-DG | |
|---|---|---|---|---|---|---|---|---|
| ID | Virus | Cells | Plates | cells seeded | M.O.I | Post-treatment | (mM) | TCID50/ml |
| 13 | SARS-CoV-2 | Vero-E6 | 24-well plate | 9.00E+04 | 0.001 | Treatment (0 h.p.i) | 45 | 3.16E+00 |
| 18 | SARS-CoV-2 | Vero-E6 | 24-well plate | 9.00E+04 | 0.001 | Treatment (0 h.p.i) | 15 | 3.16E+02 |
| 20 | SARS-CoV-2 | Vero-E6 | 24-well plate | 9.00E+04 | 0.001 | Treatment (0 h.p.i) | 5 | 1.47E+05 |
| 23 | SARS-CoV-2 | Vero-E6 | 24-well plate | 9.00E+04 | 0.001 | Treatment (0 h.p.i) | 1.67 | 1.47E+07 |
| 25 | SARS-CoV-2 | Vero-E6 | 24-well plate | 9.00E+04 | 0.001 | Treatment (0 h.p.i) | 0.56 | 3.16E+07 |
| 29 | SARS-CoV-2 | Vero-E6 | 24-well plate | 9.00E+04 | 0.001 | Treatment (0 h.p.i) | 0.19 | 6.81E+06 |
| 33 | SARS-CoV-2 | Vero-E6 | 24-well plate | 9.00E+04 | 0.001 | Treatment (0 h.p.i) | 0 | 1.47E+08 |
| 35 | SARS-CoV-2 | Vero-E6 | 24-well plate | 9.00E+04 | 0.001 | Treatment (0 h.p.i) | RMD | 6.81E+00 |
| | | | | | | | (6 μM) | |
| 37 | SARS-CoV-2 | Vero-E6 | 24-well plate | 9.00E+04 | 0.001 | Post-treatment (8 | 45 | 1.47E+01 |
| | | | | | | h.p.i) | | |
| 40 | SARS-CoV-2 | Vero-E6 | 24-well plate | 9.00E+04 | 0.001 | Post-treatment (8 | 15 | 3.16E+04 |
| | | | | | | h.p.i) | | |
| 44 | SARS-CoV-2 | Vero-E6 | 24-well plate | 9.00E+04 | 0.001 | Post-treatment (8 | 5 | 3.16E+05 |
| | | | | | | h.p.i) | | |
| 47 | SARS-CoV-2 | Vero-E6 | 24-well plate | 9.00E+04 | 0.001 | Post-treatment (8 | 1.67 | 3.16E+06 |
| | | | | | | h.p.i) | | |
| 50 | SARS-CoV-2 | Vero-E6 | 24-well plate | 9.00E+04 | 0.001 | Post-treatment (8 | 0.56 | 3.16E+07 |
| | | | | | | h.p.i) | | |
| 54 | SARS-CoV-2 | Vero-E6 | 24-well plate | 9.00E+04 | 0.001 | Post-treatment (8 | 0.19 | 1.47E+07 |
| | | | | | | h.p.i) | | |
| 57 | SARS-CoV-2 | Vero-E6 | 24-well plate | 9.00E+04 | 0.001 | Post-treatment (8 | 0 | 6.81E+07 |
| | | | | | | h.p.i) | | |
| 60 | SARS-CoV-2 | Vero-E6 | 24-well plate | 9.00E+04 | 0.001 | Post-treatment (8 | RMD | 6.81E+03 |
| | | | | | | h.p.i) | (6 μM) | |

The samples were mixed with Laemmli with β-mercaptoethanol, heated at 60° C. for 5 minutes and 20 μg (as determined by BCA assay (Thermo Scientific, Cat. no. 23225)) of the proteins were applied to the gel for Western Blot analysis. Bands were visualized using Opti-4CN Substrate Kit (Bio-Rad, Cat. no. 1708235).

Antibodies that were used in the study:
anti-SARS-CoV-2 Spike Glycoprotein S1 antibody (Abcam, Cat. no. ab275759), dilution 1:500
anti-Actin antibody, clone C4 (Sigma-Aldrich, Cat. no. MAB1501), dilution 1:4000
anti-rabbit IgG-HRP (Santa Cruz Biotechnology, Cat. no. sc-2357), dilution 1:4000
anti-mouse IgG-HRP (Santa Cruz Biotechnology, Cat. no. sc-516102), dilution 1:4000

Results and conclusions: The experiment shows that 2-DG inhibits glycosylation of spike protein in lysates of SARS-CoV-2 virus infected Vero-E6 cells (FIGS. 9a-9b). In FIGS. 9a-9b the dose-response relationship is clearly visible (selected concentrations for this blot ranges from 0.19 mM to 45 mM).

### Example 9. 2-Deoxy-D-Glucose (2-DG) Uptake Measurement in Lung Tissue Lysates of Mice Treated with 2-DG for Different Time Periods

Procedure

The analysis involved six lung tissue sections of mice (both male and female) treated by inhalation with 2-DG at a concentration of 30 mM thrice a day for 24 hours, 7 days and two weeks. The tissues were weighed, suspended in 10 mM Tris-HCl lysis buffer (pH 8.0) and then subjected to homogenization with the use of homogenizer (MPW-302, Precision Mechanics). Each analyzed sample is described in table below:

| | | Weight |
|---|---|---|
| Sample ID | Description | [mg] |
| 24DGF3 | female no. 3; inhalation treatment of 30 | 42.7 |
| | mM | |
| | 2-DG thrice a day for 24 h | |
| 24DGM1 | male no. 1; inhalation treatment of 30 mM | 49.3 |
| | 2-DG thrice a day for 24 h | |
| 7DGF2 | female no. 2; inhalation treatment of 30 | 37.1 |
| | mM | |
| | 2-DG thrice a day for 7 days | |
| 7DGM1 | male no. 1; inhalation treatment of 30 mM | 43.0 |
| | 2-DG thrice a day for 7 days | |
| 14DGF1 | female no. 1; inhalation treatment of 30 | 37.1 |
| | mM | |
| | 2-DG thrice a day for 14 days | |
| 14DGM1 | male no. 1; inhalation treatment of 30 mM | 44.1 |
| | 2-DG thrice a day for 14 days | |

As a negative control, two normal cell lines were used—BALB 3T3/c and human dermal fibroblasts.

The 2DG6P accumulation in cells was determined with the use of 2-Deoxyglucose (2-DG) Uptake Measurement Kit (Cat. No. CSR-OKP-PMG-K01TE; Cosmo Bio Co., LTD., Tokyo, Japan) according to the manufacturer's protocol.

1. Cell and tissue lysates were heat treated at 80° C. for 15 min and then centrifuged at 4° C., 15 000×g for 20 min.

2. The supernatants were transferred to new tubes and used as unknown samples for measurement method (point 5).

3. The 2DG6P standards were prepared by serial dilution of 1 mM 2DG6P in 1× Sample Diluent Buffer in the following ranges: 0; 0.3125; 0.625; 1.25; 2.5 and 5 μM.

4. 60 μL of Reagent Mix A (including NAD and low glucose-6-phosphate dehydrogenase (G6PDH)) were added to each well of 96-ell plate.

5. Then 2DG6P standard and unknown samples (20 μL) were added to each well and incubated for over 19 hours at room temperature.

6. 5 μL of Solution B (acid solution) were added to each well and incubated at 37° C. for 1 h.

7. 5 μL of Solution C (acid neutralizing solution) were added to each well and incubated at RT for 10 min.

8. 30 μL of Reaction Mix D (including NADH and high G6PDH) were added to each well and incubated at 37° C. for 1 h.

9. 5 μL of Solution E (alkali solution) were added to each well and incubated at 70° C. for 1 h and then chilled on ice for 5 min.

10. 5 μL of Solution F (alkali neutralizing solution) were added to each well and incubated at RT for 15 min.

11. 70 μL of Enzyme Cycling Solution (including glutathione disulfide (GSS) and glucose 6-phosphate (G6P), high G6PDH and glutathione reductase (GR)) were added to each well.

12. The optical density (OD) was read at 420 nm in every 2.5 min over a period of 30 min using a microplate reader preheated to 30° C.

The 96-well plate template was prepared as below and included: 2DG6P standards (B2-G2, samples (B3-G3), BALB 3T3 (B4) as well as fibroblasts (C4).

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | |
|---|---|---|---|
| A | | | |
| B | BLANK | 24DGF3 | NC1 |
| C | 0.3125 | 24DGM1 | NC2 |
| D | 0.625 | 7DGF2 | |
| E | 1.25 | 7DGM1 | |
| F | 2.5 | 14DGF1 | |
| G | 5 | 14DGM1 | |
| H | | | |

Results and Conclusions:

Temporal changes of OD for all the samples included in the analysis (FIG. 10) show that 2-DG was accumulated in the lungs of mice in animal model study due to the inhalation. Obtained data shows a plateau effect in 2-DG accumulation.

In the further aspect of the application, a formulation comprising a 2-DG (as an active ingredient) encapsulated in a dry particles composed from different excipients releasing 2-DG in the respiratory tract or forming liposomes having extended and consciously controlled the release time of the substance to several hours. By varying the ratio of DMPC to DPPC or changing lipid composition by addition varying amount of cholesterol to liposomal bilayer composed from natural soy or sunflower lecithin of the pharmaceutical purity.

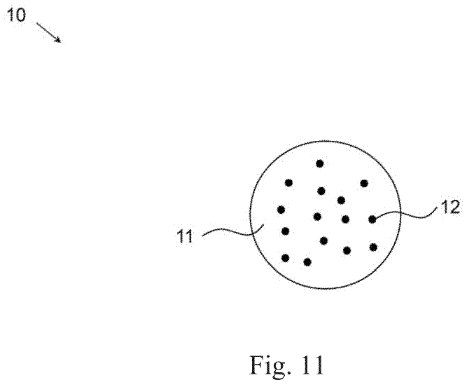

To prepare inhalable particles containing 2-DG a spray drying procedure can be applied (FIG. 11). The particles of the size lower than 5 μm can according to the application penetrate lower parts of respiratory track. The production of fine particles composed solely from 2-DG is very difficult because 2-DG has a low melting temperature and is hygroscopic. As excipients used for 2-DG particles preparation by spray-drying method mannitol, trehalose or amino acids (leucine, glycine) can be used. The spray-drying method of producing very small particles involves preparing a solution of 2-DG in water with various ingredients such as mannitol or trehalose and amino acids such as leucine or glycine. A solution containing from 0.1 to 5% solids in the solution may then be spray dried Amino acids such as leucine or glycine form a hydrophobic shell surrounding particles containing 2-DG and mannitol. This allows to solve the problem of sticking of particles prepared from the 2-DG alone.

### Example 10

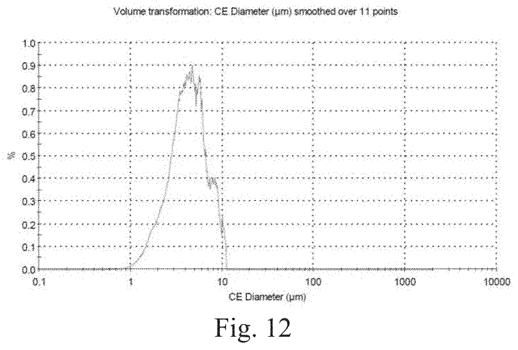

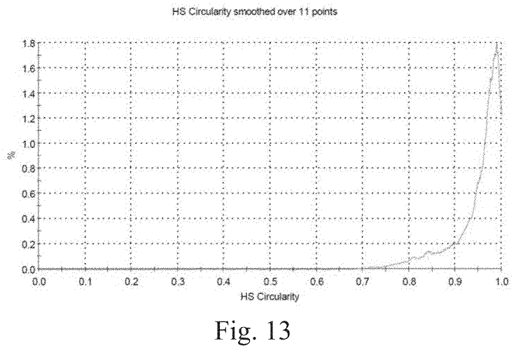

200 mg of 2-DG, 300 mg of leucine and 500 mg of mannitol were dissolved in 100 mL of deionized water. The resulting solution was spray-dried at a temperature of 150 degrees Celsius using a Mini Spray-dryer Büchi 290 device. The particles obtained have a size of about 4.6 micrometers and a very high roundness (FIG. 11, FIG. 12, FIG. 13).

### Example 11

200 mg of 2-DG, 300 mg of leucine and 500 mg of trehalose were dissolved in 100 mL of deionized water. The resulting solution was spray-dried at a temperature of 150 degrees Celsius using a Mini Spray-dryer Büchi 290 device. The particles obtained have a size of about 5 micrometers and a very high roundness.

### Example 12

200 mg of 2-DG, 800 mg of trehalose and 100 mg of leucin were dissolved in 100 mL of deionized water. The resulting solution was spray-dried at a temperature of 100 degrees Celsius using a Mini Spray-dryer Büchi 290 device. The particles obtained have a size of about 4 micrometers and a very high roundness.

### Example 13

200 mg of 2-DG, 800 mg of mannitol and 100 mg of leucin were dissolved in 100 mL of deionized water. The resulting solution was spray-dried at a temperature of 100 degrees Celsius using a Mini Spray-dryer Büchi 290 device. The particles obtained have a size of about 4 micrometers and a very high roundness.

### Example 14

200 mg of 2-DG, 200 mg of DPPC and DMPC 64:36 mol/mol ratio, 400 mg of leucine and 200 mg of mannitol were dissolved in 100 mL of 60% ethanol at 50° C. The mixture was spray dried in a Mini Buchi machine and the obtained proliposome particles had size of about 4 μm. The particles were mixed with 37° C. physiological saline to obtain liposomes with an average size of 2 μm. At time t=0, it was determined that about 60% of the active substance was released, the rest was slowly released within 18 hours.

### Example 15

200 mg of 2-DG, 300 mg of DPPC and DMPC (70:30 mol/mol), 400 mg of leucine and 100 mg of trehalose were dissolved in 100 mL of 60% ethanol at 50° C. The mixture was spray dried in a Mini Buchi machine and the obtained proliposome particles had size of about 4.5 μm. Proliposomes particles were mixed with 37° C. physiological saline to obtain liposomes with an average size of 2.7 μm. At time t=0, it was determined that about 50% of the active substance was released, the remaining amount was released over the next 8 hours.

### Example 16

200 mg of 2-DG, 300 mg of DPPC and DMPC (70:30 mol/mol), 300 mg of glycine and 200 mg of trehalose were dissolved in 100 mL of 60% ethanol at 50° C. The mixture was spray dried in a Mini Buchi machine and the obtained proliposome particles were mixed with 37° C. physiological saline to obtain liposomes with an average size of 3 μm. At time t=0, it was determined that about 45% of the active substance was released, the remaining amount was released over the next 8 hours.

### Example 17

200 mg of 2-DG, 300 mg of SPC/Chol (70:30 mol/mol), 300 mg of urea and 200 mg of leucin were dissolved in 100 mL of 50% tert-butanol at 50° C. The mixture was spray dried in a Mini Buchi machine and the obtained proliposome particles were mixed with 37° C. physiological saline to obtain liposomes with an average size of 1.2. At time t=0, it was determined that about 65% of the active substance was released, the remaining amount was released over the next 8 hours.

### Example 18

900 mg of DPPC and DMPC (70:30 mol/mol) were dissolved in 3 mL of ethanol at 50° C. and then 10 mL of water with 0.5 g of 2-DG dissolved were purred in to achieve oligolamellar liposomes. The suspension was then extruded 6 times through 200 nm polycarbonate filter in a thermobarrel extruder set at 40°. The resulted unilamellar liposomes were then incubated 15 min at 70° C. to increase 2-DG encapsulation. The liposomal suspension was next cooled down to 40° C. and diluted with water solution containing trehalose and leucine. Final lipid concentration was 20 mg/mL, 5% trehalose and 1% leucine concentration.

After spray-drying the particle size was 4.5 μm, the 41% of the encapsulated 2-DG remained encapsulated after reconstitution of the particles in 0.9% NaCl at 37° C. Liposomes size remained essentially unchanged and was 186 nm.

### Example 19

200 mg of DPPC and DMPC (70:30 mol/mol) were dissolved in 4 mL of ethanol/propylene glycol mixture (1:1) at 50° C. This solution was then mixed with 1 mL of the solution containing 50 mg 2-DG and clear solution was achieved. This solution forms liposomes when mixed with 150 mM NaCl solution at 37° C. The encapsulation efficiency of the 2-DG is near 72%.

### Example 20

1 g of lipids of the composition HSPC/DSPG/Chol/DSPE-PEG 2000 (55:10:30:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 ml distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 36%, the liposomes size was 110 nm (FIG. 14).

### Example 21

1 g of lipids of the composition SM/Chol (55:45, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded 4 times through 400 nm and then 8 times through 80 nm polycarbonate filter on the thermobarrel extruder in order to achieve large unilamellar liposomes. To the liposomal suspension ethanol was slowly pipetted to achieve 30% concentration. The liposomal suspension was next incubated to 75° C. for 10 min. The encapsulation efficiency of the 2-DG was 27%, the liposomes size was 115 nm.

### Example 22

1 g of lipids of the composition HSPC/Chol/DSPE-PEG 2000 (65:30:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 ml distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 31%, the liposomes size was 113 nm.

### Example 23

1 g of lipids of the composition DPPC/Chol/DSPE-PEG 2000 (65:30:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 ml distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 29%, the liposomes size was 103 nm.

### Example 24

1 g of lipids of the composition DPPC/DPPG/Chol/DSPE-PEG 2000 (55:10:30:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 31%, the liposomes size was 107 nm.

### Example 25

1 g of lipids of the composition HSPC/Chol/DSPE-PEG 2000 (65:30:5, mol/mol) was dissolved in 10 mL of cyclohexane and freeze in liquid nitrogen. The resulting ice was subsequently freeze-dried and dry lipid cake was suspended in 20 mL of solution of 200 mM 2-DG and 200 mM sodium ascorbate at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 27%, the liposomes size was 109 nm.

### Example 26

1 g of lipids of the composition DPPC/Chol/DSPE-PEG 2000 (65:30:5, mol/mol) was dissolved in 10 mL of cyclohexane and freeze in liquid nitrogen. The resulting ice was subsequently freeze-dried and dry lipid cake was suspended in 20 mL of solution of 200 mM 2-DG and 200 mM sodium ascorbate at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 26%, the liposomes size was 108 nm.

### Example 27

1 g of lipids of the composition HSPC/DSPG/Chol/DSPE-PEG 2000 (55:10:30:5, mol/mol) was dissolved in 10 mL of cyclohexane and freeze in liquid nitrogen. The resulting ice was subsequently freeze-dried and dry lipid cake was suspended in 20 mL of solution of 200 mM 2-DG and 200 mM sodium ascorbate at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 27%, the liposomes size was 116 nm.

### Example 28

1 g of lipids of the composition DPPC/DPPG/Chol/DSPE-PEG 2000 (55:10:30:5, mol/mol) was dissolved in 10 mL of cyclohexane and freeze in liquid nitrogen. The resulting ice was subsequently freeze-dried and dry lipid cake was suspended in 20 mL of solution of 200 mM 2-DG and 200 mM sodium ascorbate at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 24%, the liposomes size was 102 nm.

### Example 29

1 g of lipids of the composition SM/Chol (55:45, mol/mol) was dissolved in 10 mL of cyclohexane and freeze in liquid nitrogen. The resulting ice was subsequently freeze-dried and dry lipid cake was suspended in 20 mL of 200 mM 2-DG and 200 mM sodium ascorbate solution at 64° C. The multilamellar liposomes were then extruded 4 four times through 400 and then 8 times through 80 nm polycarbonate filter on the thermobarrel extruder in order to achieve large unilamellar liposomes. To the liposomal suspension ethanol was slowly pipetted to achieve 30% concentration. The liposomal suspension was next incubated to 75° C. for 10 min. The non-encapsulated 2-DG was removed by dialysis method. The encapsulation efficiency of the 2-DG was 32%, the liposomes size was 117 nm.

### Example 30

1 g of lipids of the composition HSPC/Chol/PA/DSPE-PEG 2000 (55:30:10:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 23%, the liposomes size was 101 nm.

### Example 31

1 g of lipids of the composition DPPC/Chol/PA/DSPE-PEG 2000 (55:30:10:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 22%, the liposomes size was 98 nm.

### Example 32

1 g of lipids of the composition HSPC/DSPG/Chol/PA/DSPE-PEG 2000 (35:20:30:10:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 34%, the liposomes size was 99 nm.

### Example 33

1 g of lipids of the composition DPPC/DPPG/Chol/PA/DSPE-PEG 2000 (35:20:30:10:5, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded trough 400 and then 100 nm polycarbonate filter on the thermobarrel extruder in order to achieve large unilamellar liposomes. The resulting liposomes were then frozen and freeze-dried. Liposomal powder was then rehydrated by addition of 10 mL distilled water at 64° C. The oligolamellar liposomes were next extruded trough 80 nm polycarbonate filter on thermobarrel extruder. The non-encapsulated 2-DG was removed by dialysis method. The encapsulation efficiency of the 2-DG was 28%, the liposomes size was 95 nm.

### Example 34

1 g of lipids of the composition SM/Chol/PA/DSPE_PEG 2000 (43:35:10:0.2, mol/mol) was dissolved in 10 mL of cyclohexane and freeze in liquid nitrogen. The resulting ice was subsequently freeze-dried and dry lipid cake was suspended in 20 mL of 600 mM solution of the 2-DG at 64° C. The multilamellar liposomes were then extruded 4 four times through 400 and then 8 times through 80 nm polycarbonate filter on the thermobarrel extruder in order to achieve large unilamellar liposomes. To the liposomal suspension ethanol was slowly pipetted to achieve 30% concentration. The liposomal suspension was next incubated to 75° C. for 10 min. The non-encapsulated 2-DG was removed by dialysis method.

The encapsulation efficiency of the 2-DG was 23%, the liposomes size was 113 nm.

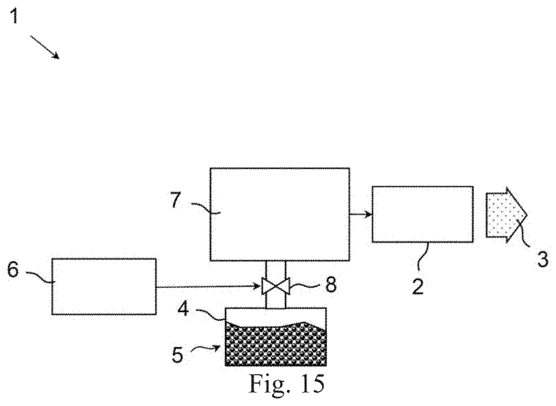

FIG. 15 shows a schematic view of a device for inhaling a substance according to an embodiment. The device 1 comprises a discharge nozzle 2 and a container 4 for receiving and keeping the substance 5 as well as an actuator 6 for activating the device 1. The actuator 6 is configured to release a certain amount or dose of the substance 5 which is kept in the container 4 for transferring the substance 5 through the dis-charge nozzle 2 of the device 1. In the embodiment of FIG. 15, the device 1 comprises an air flow channel 7 or chamber for conveying the substance 5 released by the actuator 6 to the discharge nozzle 2. The substance 5 in the container 4, shown in FIG. 15 is a powder comprising a carrier material and an active ingredient out of a group comprising in particular 2-DG.

In some embodiments, the container 4 is configured to keep a substance in liquid form. The substance 5 may, in particular, comprise a liquid out of the group comprising water, alcohol, liquid glucose, or aqueous solution, in which the active agent is contained.

In some embodiments, the substance 5 in the container 4 is in the form of powder of particles with lactose and/or liposome. Lactose or liposome can serve as a carrier for the active ingredient, such that they can be easily transferred or delivered to the human body. In some embodiments, the active agent is encapsulated in liposomes, in order to achieve a longer or delayed effect in the human body, thus increasing the duration of the therapeutic or prophylactic effect. Particles may also comprise cholesterol which stabilizes liposomes such that an even stronger delay of the agent can be achieved. The particles may comprise a mixture of different liposome. In particular, a mixture of small liposomes, with an average size of less than 100 nm and large liposomes, with an average size of more than 150 nm. By providing different liposome sizes, a desired time profile of the active agent activity can be achieved.

The Device 1 may comprise a chamber or reservoir for a propellant, such as CFC (chlorofluorocarbon) and/or HFA (hydrofluoroalkane), for propelling the substance along the flow channel towards the nozzle. The propellant can in particular, facilitate the delivery and dosage of the active ingredients.

In the embodiment of FIG. 15, the device 1 comprises a dosage valve 8 arranged at an outlet of the container 4 and the actuator 6 may be functionally connected to the dosage valve 8 and be configured to activate the dosage valve 8. The dosage valve may be configured to release a defined amount of the substance 5, which is to be released each time when the activator 6 is activated. By means of the dosage valve 8, a precise dos-age of the substance 5, in particular, of the active ingredient or agent comprised in the substance 5 can be achieved. The dosage valve 8 may be an adjustable dosage valve such that, prior to dispensing the substance, the dosage of the substance can be adjusted.

The dosage valve 8 can be, in particular, configured to keep the amount of the active agents in the released portion of the substance 5 and the dosage in the range of 5 to 10 millimoles. By limiting the amount of the active agent in the particles, side effects related with too high dosage can be avoided. In some embodiments, the liposomes have a transition temperature, from solid to liquid, in the range from 35° C. to 45° C., more specifically, between 37° C. and 40° C. degrees about 37° C. Thus, the liposomes may easier dissolve after the substance has been applied to the human body.

The air flow channel 7 may be configured to support turbulences in the air flow. The turbulences in the air flow can facilitate entraining the particles released from the container and propel them towards the discharge nozzle 2 of the device 1. Further, due to the turbulences in the air flow, the phase space occupied by the particles released from the container 4 can be increased such that a broad distribution of the resulting particle jet can be achieved. The broad distribution of the particles may be particularly helpful to avoid local overdoses of the active ingredients at the human tissues exposed to the substance.

In some embodiments, the airflow channel 7 is configured such that the air flow in the air flow channel can be created by the user by inhaling the air while keeping the discharge nozzle 2 of the device in a nostril or in the mouth. Such a device does not require any additional source of energy for providing the air flow.

The actuator 6 can be configured to provide a pressurized air flow in the air flow channel. The pressurized air released by the actuator can, in particular, support turbulences which can help to entrain the substance particles located at the outlet of the container 4 when the dosage valve 8 is open.

FIG. 11 shows a compound particle according to an embodiment.

The particle 10 comprises a carrier material 11 with an active ingredient 12, wherein the active ingredient 12 is encapsulated or integrated in the carrier material 11. In this embodiment, the particle is formed by spray drying and the active ingredient 12 comprises 2-DG. In some embodiments the carrier material may comprise trehalose and/or phospholipids. The active ingredient may comprise one or more different types of agents out of the group comprising ribavirin, emetine, 2-DG and NMS-873. These active ingredients can serve as translation inhibitors for preventing or suppressing viral replication and/or as agents for suppressing the growth and reproduction of the host cells attacked by viruses. Due to prevention of the viral replication and suppressing the growth and reproduction of the host cells, these active ingredients can serve not only as a medication against viral-infectious diseases but also as a prophylaxis for preventing a viral attack or contagion of the human body. In some embodiments, the carrier material may comprise a mixture of different liposomes showing different stability and transition temperature characteristics.

FIG. 14 shows a compound particle according to an embodiment. The particle 10, similar to the particle of FIG. 11, is a compound particle comprising a carrier material 11 (liquid) with an active ingredient 12. However, in this embodiment, the carrier material 11 and the active ingredient 12 is surrounded by lipid by-layer 13. The active ingredient 12 may comprise one or more different types of agents and in particular comprises 2-DG.

The carrier material 11, in this embodiment, is a liquid. The carrier material may comprise a liquid out of the group comprising water, liquid glucose or aqueous solution. In some embodiments, the carrier material comprises an aqueous solution of one or more salt. In some embodiments, the salt in the aqueous solution is NaCl. The salt concentration in the aqueous solution may vary in the range from 1% to 5%, in particular, 1.5% to 2.5%, or from 1.8% to 2.2%. In some embodiment, the aqueous solution comprises an additive for regulating the pH value of the aqueous solution. By regulating the pH value in the aqueous solution, the stability of the compound particle, in particular, the shell 13 of the compound particle, can be adjusted such that a desired sustained release of the active agent in the human body can be achieved.

The shell 13 encapsulating the carrier material 11 and the active agent 12 may comprise lipid by-layer composed of phospholipids, PEGylated phospholipids, sterols and surfactants. In some embodiments, the shell 13 may comprise a mixture of different phospholipids showing different stability and transition temperature characteristics.

The size of the particles of FIG. 11 may vary from in a range 0.5-5 μm and the size of the particles of FIG. 14 may vary from in a range 50 nm-1000 nm. —Such fine particles 10 can be easily applying to a target region of the human body, in particular, by spreading them as aerosol particles or powder particles.

Further, liposomes with different properties can be used. Thus, by choosing different size and/or properties of liposomes in the shell of the compound particles, a desired time profile for releasing the active agent in the human body can be achieved. The liposome encapsulation can help to reduce toxic reactions or irritations, when the substance is applied to the human body. In particular, the during inhalation, the liposome shell covers the carrier material with the active agent, such that the active agent can get to deeper zones of the respiratory tract without getting in direct contact with the tissues, before reaching the target location. Thus, unnecessary exposure of the human tissue to the active agent, in particular in the regions unaffected by the virus, can be avoided.

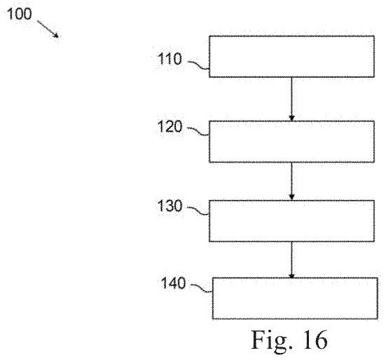

FIG. 16 shows a flow chart of a method of dispensing a substance according to an embodiment. According to the method 100, in step 110, a substance in the form of aerosol particles or powder particles is provided. The provided particles may be kept, in particular, in a container of a device for inhaling the substance. Further, in step 120, an aerosol is created, the particles being suspended in the aerosol. The aerosol can be created, in particular, in an air flow channel or chamber of the device for inhaling the substance.

Further, in step 130, a directed flow of the aerosol is created such that the suspended particles move essentially along the flow direction of the aerosol. The particles may comprise at least one active ingredient or active agent out of the group comprising ribavirin, emetine, 2-DG and NMS-873. The method further comprises directing 140 the directed flow or jet of the aerosol towards target areas of the human body for dispensing the substance. In some embodiments, the target areas may comprise a throat or nasal area, pharynx or nasal mucosa as well as lung tissue of humans. By discharging the sub-stance with the active ingredients, viral activity can be affected.

By discharging translation inhibitors in the target areas, viral replications in the human cells can be prevented or suppressed. Furthermore, by suppressing the viral replication in the early stage, later pathological consequences can be avoided as well. Thus, the method can also serve as a prophylaxis for avoiding viral diseases.

Even though, the mechanisms are not completely understood, 2-DG molecules, as compared to glucose, are characterized by stability against cellular metabolism, in particular, in cancerous or virally compromised cells. Because of the similarity with glucose molecules, cells may regard 2-DG molecules as glucose molecules and capture them. Within the cells, 2-DG molecules can even undergo phosphorylation. The resulting 2-DG-6-phosphate, however, in contrast to phosphorylated glucose, does not further participate in glycolysis. Thus, 2-DG-6-phophate can remain in a host cell, in particular, in a host cell attacked by a SARS-CoV-2 virus without undergoing glycolysis and, hence, without producing energy which is necessary for cellular activities including biogenesis and reproduction of host cells. The viruses do not have their own metabolism. Instead, the viruses can penetrate into healthy cells or host cells and modify their DNA such that these cells start to produce more viruses and to reproduce themselves as well, resulting in a multiplicity of infected cells which can produce and disseminate more viruses. Similar to cancerous cells, the host cells also require vast amount of energy, in particular, for growth and production of further viruses.

Some viruses, in particular the SARS2 viruses (from the Corona family), are transmitted via the respiratory tract. They nest in the frontal sinuses and lungs of the infected patients.

Therefore, by applying the growth and multiplication-inhibiting 2-DG directly to the airways (right where the infecting host cells reside), are helpful both for prophylaxis and therapy of the diseases caused by the viruses. Further, by providing the active agent, in particular 2-DG, in the form of very finely ground powder or particles which may be inhaled by means of an inhaler (Formulation 1) such that the growth and reproduction of the cells in the respiratory tract, infected by SARS-CoV-2 can be suppressed. By adding additional substances, in particular matrix material, to the powder or using a liquid (suspension) form (SprayFormulation2) the side effects can be reduced and/or absorption rate in the organism can be control the absorption rate can be improved. In particular, some therapeutic substances, in particular, 2-DG, is the short half-life or dwelling time in the organism. In some cases, the active ingredients, e.g. 2-DG, are not detectable in the blood after approximately 48 to 53 minutes after the administration. There are indications that the active sub-stances can quickly decay or enter into reaction with other substances and still remain active. In the body, 2-DG can be detected by a number of alternating defense and excretion mechanisms and can thus trigger reactions which convert or change it very quickly into other substances. By enclosing the active ingredients, in particular 2-DG, in liposomes, or structures with lipids, before applying them through the respiratory tract, the time profile of their activity in the organism can be influenced, in particular while fighting SARS-CoV-2 virus. The liposomes can be mixture of different liposomes configured such that they circulate for several hours without the substance encapsulated or integrated in the liposome becoming detected by the defense mechanisms of the organ-ism. In some embodiments, the liposome is configured such that an essentially constant concentration of the active ingredient, in particular 2-DG, in the blood is maintained during many hours, in some embodiments, even more than 100 hours.

The substance or formulation with liposomes, in particular, liposome encapsulation, also reduces mechanical irritations in the respiratory tract and reactions, such as coughing, sneezing, rash, toxic consequences, allergies etc. The liposomes can also facilitate to reach the target locations, such as lungs, which is crucially important for precise targeting of the active ingredients. Further, liposome pH values can be programmed or adjusted according to specific application, in particular, in accordance with the properties of the target tissue. The adjustment of the pH value can also influence the sustained release, activity and mobility of the formulation. Liposomes can, in particular, influence the rate of release of the active agent, in particular 2-DG, after administration.

Thus, the frequency of usage of the inhaler for administration of the substance can be reduced. Furthermore, due to the local delivery of 2-DG in small quantities, the overall load of the medication in the organism, can be reduced, resulting in reduction of possible side effects. Further, a combination of active ingredients can be applied locally and/or non-locally. In particular, 2-DG can be combined with one or more further active agents, such that a synergetic effect in fighting the viral infection is achieved.

### REFERENCE SYMBOLS AND NUMERALS

1 Device
2 discharge nozzle
3 particles
4 container
5 substance
6 actuator
7 flow channel
8 dosage valve
10 particle
11 carrier material
12 active ingredient
13 shell
100 method
110 method step
120 method step
130 method step
140 method step

Below some of the above and further embodiments, in particular of the further aspects of the application, are further defined in the following list of embodiment items. In this list, the term “substance” in particular relates to further embodiments of a medical preparation comprising 2-DG for use in a medical method to prevent and/or treat Covid 19—albeit some other substances or active ingredients might be mentioned in these embodiments, too.

Item 1. Method of applying a substance to a human body, the method comprising:
providing the substance, and
delivering the substance in the form of aerosol particles or powder particles to the nose or mouth of a person, wherein the particles comprise at least one active ingredient out of the group comprising ribavirin, emetine, 2-DG and NMS-873.

Item 2. The method according to embodiment Item 1, wherein the particles comprise a carrier material carrying the active ingredient.

Item 3. The method according to embodiment Item 2, wherein the carrier material comprises a liquid out of the group comprising water, alcohol, propylene glycol, glycerol, liquid glucose, or aqueous solution.

Item 4. Method according to one of the previous embodiments Items, wherein the particles comprise at least a lactose and/or liposome.

Item 5. Method according to one of the previous embodiments Items, wherein the delivering of the substance comprises propelling the substance by means of at least one propellant comprising CFC (chlorofluorocarbon) and/or HFA (hydro-fluoroalkane).

Item 6. Use of a substance for inhalation, wherein the substance is provided in the form of aerosol particles or powder particles, and wherein the particles comprise at least one active ingredient out of the group comprising ribavirin, emetine, 2-DG and NMS-873, wherein the sub is delivered to the mouth or nose of a person.

Item 7. Method of dispensing a substance, the method comprising:
providing the substance in the form of aerosol particles or powder particles,
creating an aerosol with the particles suspended in the aerosol,
creating a directed flow of aerosol such that the suspended particles move essentially along the flow direction of the aerosol, wherein the particles comprise at least one active ingredient or active agent out of the group comprising ribavirin, emetine, 2-DG and NMS-873, and directing the directed flow or jet of the aerosol towards target areas of the human body for dispensing the substance.

Item 8. Device for inhaling a substance in the form of aerosol particles or powder particles, the device comprising:
a discharge nozzle for dispensing the substance in the form of aerosol particles or powder particles,
a container for receiving and keeping the substance,
and
an actuator for activating the device, the actuator being configured to release a certain amount or dose of the substance kept in the container for conveying the substance through the discharge nozzle of the device, wherein the particles comprise at least one active ingredient out of the group comprising ribavirin, emetine, 2-DG and NMS-873.

Item 9. The device according to embodiment Item 8, wherein the actuator is a manual actuator which can be activated manually.

Item 10. The device according to embodiment Items 8 or 9, wherein device comprises a dosage valve defining the amount of the substance to be released, and wherein the actuator is configured to activate the dosage valve.

Item 11. The device according to embodiment Item 10, wherein the dosage valve is an adjustable valve such that the dosage of the substance can be adjusted prior to dispensing the substance.

Item 12. The device according to one of the embodiments Items 8 to 11, wherein upstream to the discharge nozzle, an air flow channel for conveying the substance released by the actuator to the discharge nozzle is arranged.

Item 13. The device according to one of the embodiments Items 8 to 12, wherein the air flow in the air flow channel can be created by the user by inhaling the air while keeping the nozzle of the device in a nostril or in the mouth.

Item 14. The device according to one of the embodiments Items 8 to 13, wherein the actuator is further configured to provide a pressurized air flow in the airflow channel.

Item 15. The device according to one of the embodiments Items 8 to 14, wherein the device further comprises a reservoir with a propellant, and wherein the actuator is further configured to release the propellant such that the substance can be conveyed by droplets of propellant along the flow channel.

Item 16. Substance for applying to a human or animal body for the treatment of lung tissue cells by inhalation, the substance comprising at least one active ingredient out of the group comprising ribavirin, emetine, 2-DG and NMS-873.

Item 17. The substance according to one of the embodiments 16 to 19 for use in the therapy of COVID-19.

Item 18. The substance according to embodiments Items 16 or 17, wherein the substance comprises non-toxic concentration of the active ingredient for preventing SARS-CoV-2 replication in human lung tissue cells or in human nasal mucosa cells.

Item 19. The substance according to one of the embodiments Items 16 to 18, wherein the substance comprises a carrier material carrying the active ingredient.

Item 20. The substance according to embodiment Item 19, wherein the carrier material comprises a liquid out of the group comprising water, alcohol, liquid glucose, or aqueous solution.

Item 21. The substance according to one of the embodiments 16 to 20, wherein the substance comprises lactose and/or liposome.

Below some of the above and further embodiments, in particular of the further aspects of the application, are further defined in the following second list of embodiment items. In this second list, the term “substance” in particular relates to further embodiments of a medical preparation comprising 2-DG for use in a medical method to prevent and/or treat Covid 19—albeit some other substances or active ingredients might be mentioned in these embodiments, too.

Item 1. 2-Deoxy-D-Glucose (2-DG) for use in a medical method to prevent and/or to treat Covid-19, wherein 2-DG is provided as a preparation in an amount and a formulation that results in an effective tissue concentration to partially or completely inhibit glycosylation of a SARS-CoV-2 spike protein.

Item 2. 2-Deoxy-D-Glucose (2-DG) for use in a medical method to prevent and/or to treat a viral disease caused by an enveloped virus comprising a spike protein, wherein 2-DG is provided as a preparation in a liposomal or a proliposomal formulation.

Item 3. 2-Deoxy-D-Glucose (2-DG) for use in a medical method to prevent and/or to treat Covid-19 by inhalation

Item 4. 2-DG according to one of the previous items,
wherein 2-DG is provided as a micron or a submicron particle, wherein in particular said micron or submicron particle is a mechanically micronized particle or is a micronized particle obtained by spray drying.

Item 5. 2-DG for the use according to one of the previous Items,
wherein 2-DG is provided at an effective tissue concentration to inhibit at least 30%, in particular at least 50%, 70%, 80% 90%, 95% or 99% of the glycosylation the SARS-CoV-2 spike protein.

Item 6. 2-DG for the use according to Item 4,
wherein the effective tissue concentration is selected in a range of 0.1 mM up to 25 mM, in particular wherein the tissue is a respiratory tissue.

Item 7. 2-DG for the use according to one of the previous Item,
wherein 2-DG is provided as a preparation in a liposomal or proliposomal formulation and
wherein the preparation comprises an amount of 2-DG in a range of 1% and 75% w/w of the total weight of the preparation, in particular an amount of 2-DG in a range with lower limit of 10% or 20% or 30% and an upper limit of 35% or 45% to 55 w/w of the total weight of the preparation, in particular between 10% and 40% w/w or between 15% w/w and 30% w/w of the total weight of the preparation,
wherein the preparation further comprises an excipient comprising a lipid fraction comprising or consisting of a phospholipid fraction in an amount of 5% to 80% w/w, in particular 15% to 50% w/w of the total weight of the preparation
characterized in that,
the total the phospholipid fraction comprises at least 10% w/w up to 60% w/w, preferably in a range between 20% w/w and 40% w/w most preferably in a range between 30% w/w and 50% w/w of a combination of dipalmitoyl phosphatidylcholine (DPPC) and dimyristoylphosphatidylcholine (DMPC) in any weight ratio.

Item 8. 2-DG for the use according to one of the previous Item,
wherein 2-DG is provided as a preparation in a in a liposomal or proliposomal formulation and
wherein the preparation comprises DPPC and DMPC in a molar ratio from 50:50 to 90:10, preferably a molar ratio of DPPC to DMPC from 60:40 to 75:25 molar ratio, most preferably from 65:35 to 71:29 molar ratio (phase transition temperature ranging from 35 to 36.3° C.)

Item 9. 2-DG for the use according to one of the previous items,
wherein 2-DG is provided as a preparation in a liposomal or proliposomal formulation and
wherein the preparation comprises a further excipient selected from the group of excipients comprising
an amino acid, in particular leucine or glycine, in particular in an amount of 0 w/w up to 50 w/w of the total weight of the preparation, more particular in an amount of 5% w/w up to 30% w/w;
trehalose in an amount of 0 w/w up to 60% w/w of the total weight of the preparation, more particular in an amount of, 5% w/w up to 30% w/w;
mannitol, 0% w/w up to 60% w/w of the total weight of the preparation, more particular in an amount of 5% w/w up to 30% w/w;
one or more further phospholipid, in particular a natural or a semi-synthetic phospholipid,
one or more further negatively or a positively charged phospholipid, in particular in an amount of 1% up to 10% of the molar % of the phospholipid fraction, more particular in an amount of 5 molar % up to 10 molar %, wherein in particular the one or more further phospholipid is in particular selected from the group comprising phosphatidylglycerol, dimyristoyl phosphatidylglycerol, dipalmitoylphosphatidylglycerol, hydrogenated soybean phosphatidylcholine (HSPC), soybean phosphatidylcholine (SPC) and wherein optionally the phospholipids comprises DPPE or DSPE with covalently attached hydrophilic polymer, in particular a PEG or polyglycerol in a molar ratio of 0 to 10 mole % of the total lipid fraction, more particular in an amount of 5 molar % of total lipid fraction.
sterol, in particular cholesterol in an amount of 0 molar % up to 55 molar % of the total the lipid fraction, more particular in an amount of 30 molar % up to 45 molar %.
nicotinic acid amide, in an amount of 10% w/w up to 80% w/w of the total weight of the preparation, more particular in an amount of 20 to 60% w/w of the preparation.
urea in an amount of 20% w/w up to 80% w/w of the total weight of the preparation, more particular in an amount of 40 to 60% w/w of the preparation.

Item 10. 2-DG for the use according to one of the previous Items,
wherein 2-DG is provided as a preparation in a liposomal or proliposomal formulation and
wherein said formulation upon contact with an aqueous environment forms liposomes within a size range selected from group of size ranges comprising
liposome sizes ranging from 30 nm to 200 nm in particular for intravenous delivery;
liposome sizes ranging from 50 nm to 5 μm, in particular for pulmonary delivery;
unilamellar liposomes of sizes ranging from 30 to 120 nm.

Item 11. 2-DG for the use according to one of the previous Items,
wherein 2-DG is provided as a preparation in a liposomal or proliposomal formulation and
with an amount of encapsulated 2-DG in a range of 10 mg to 1000 mg, in particular 50 mg to 500 mg, preferably, 100-200 mg per unit dosage.

Item 12. 2-DG for the use according to one of the previous Items,
wherein 2-DG is provided as a preparation in a liposomal or proliposomal slow release formulation in particular for intravenous administration, and
wherein the amount of the active ingredient, 2-DG, released at the time of administration (t=0) ranges from 10% to 70% w/w of the total amount of the active ingredient in the preparation and preferably ranges from 30% to 50% w/w.

Item 13. 2-DG for the use according to one of the previous Items,
wherein 2-DG is provided as a preparation in a liposomal or proliposomal slow release formulation in particular for intravenous administration, and wherein a dosage of 2-DG, in particular one unit dosage is administered at intervals between once in 2 hours to 48 hours, in particular between once in 4 to 24 hours or at intervals of approximately 6 or 12 hours.

Item 14. 2-DG for the use according to one of the previous Items,
wherein 2-DG is provided as a preparation in a liposomal or proliposomal formulation, and
wherein the liposomal or proliposomal formulation obtained by a method of preparation selected from a group of methods comprising
lyophilizing a liposomal formulation comprising 2-DG as active ingredient, wherein in particular the preparation
by spray drying a composition comprising 2-DG, phospholipids and optional further excipients,
wherein the optional further excipient is selected from the group comprising
auxiliary phospholipid for spray drying selected from natural phosphatidylglycerol, DMPG, DPPG, DSPG and natural cardiolipin used at a concentration of 0 to 30 mol % of the total phospholipid content
and/or
auxiliary lipids for spray drying selected from the group of sterols, in particular cholesterol
method of preparation of proliposomes by dehydration-rehydration a composition comprising 2-DG, phospholipids and optional excipients followed by extrusion and spray drying for the formation of unilamellar liposomes.

Item 15. 2-DG for the use according to one of the previous Items,
wherein 2-DG is provided as a preparation for administration by inhalation,
wherein the preparation comprises particles for inhalation with a diameter of 10 μm or less, in particular less than 5 μm, 3 μm, 1 μm, 0.3 μm or 0.1 μm, more particular particles with a diameter in a range with a lower limit between 0.1 μm and 1 μm and with an upper limit between 0.5 and 5 μm.

Item 16. 2-DG for the use according to one of the previous Items,
wherein 2-DG is provided as a dry preparation for administration by inhalation,
wherein the preparation comprises a content of 2-DG as active ingredient of 5% to 80% w/w of the total dry weight of the preparation, preferably 15% to 60% w/w.

Item 17. 2-DG for the use according to one of the previous Items,
wherein 2-DG is provided is formulated as a micron or a submicron particle for administration by a nebulizer,
wherein in particular 2-DG is dissolved in an isotonic solution, in particular in 0.9% saline.

Item 18. 2-DG for the use according to one of the previous Items,
wherein 2-DG is provided as a preparation for administration by inhalation,
wherein the preparation comprises an amount of 2-DG as active ingredient per unit dosage of 0.1 mg to 20 mg, in particular 0.25 mg to 10 mg, more particularly 1 to 2 mg.

Item 19. 2-DG for the use according to one of the previous Items,
wherein 2-DG is provided as a preparation for administration by inhalation as a slow release formulation,
wherein the amount of the active ingredient, 2-DG, released at the time of administration (t=0) ranges from 10% to 70% w/w of the total amount of the active ingredient in the preparation and preferably ranges from 30% to 50% w/w.

Item 20. 2-DG for the use according to one of the previous Items,
wherein 2-DG is provided as a preparation for administration by inhalation,
wherein a dosage of 2-DG, in particular one unit dosage is administered at intervals between once in 0.5 hours to 24 hours, in particular between once in 1 to 12 hours and preferably at intervals of approximately up to 2 or 4 or 6 or 8 or 10 or 12 or 24 hours.

Item 21. Ascorbic acid, Sodium, ammonium, magnesium ascorbate at the concentration from 1 to 600 mM alone or in combination with 2-DG (600 to 1 mM) encapsulated in unilamellar liposomes or proliposomes of the size from 30-250 nm.

Item 22. 2-DG for the use according to one of the previous Items,
wherein 2-DG is provided as a preparation for administration by inhalation,
wherein Ascorbyl palmitate within liposomes from 2 mol % to 60 mol % to other lipids or surfactants such like Tween 20, Tween 80, Pluronics, etc, and with 2-DG in the solution encapsulated within liposomes from 1 to 600 mM encapsulated in unilamellar liposomes of the size from 30-250 nm.
2-Deoxy-D-Glucose (2-DG) is provided for use in a medical method to prevent and/or treat a viral disease, in particular Covid-19. In one aspect, 2-DG is provided in an effective tissue concentration for partial or complete inhibition of glycosylation of a SARS-CoV-2 spike protein. In one aspect, 2-DG is provided in a liposomal or a proliposomal formulation to prevent and/or to treat a viral disease caused by an enveloped virus comprising a spike protein. In one aspect 2-DG is provided as a preparation for administration by inhalation.

While there are shown and described presently further embodiments of the application, it is to be distinctly understood that the application is not limited thereto but may be otherwise variously embodied and practiced within the scope of the following claims.

1. 2-Deoxy-D-Glucose (2-DG) for use in a medical method to prevent and/or to treat a viral infection in a subject by a virus comprising a spike protein, wherein the 2-DG is provided as a preparation in an amount and a formulation to tissue of the subject that results in an effective tissue concentration to partially or completely inhibit glycosylation of the spike protein.

2. The 2-DG according to claim 1, comprising at least one selected from the group consisting of: ribavirin, emetine, and NMS-873.

3. The 2-DG according to claim 1, wherein the effective tissue concentration inhibits at least 30% of the glycosylation of the spike protein.

4. The 2-DG according to claim 1, wherein the effective tissue concentration is in a range of 0.1 mM to 25 mM.

5. The 2-DG according to claim 1, wherein the tissue comprises respiratory tissue.

6. The 2-DG according to claim 1, wherein the virus is a Coronavirus.

7. The 2-DG according to claim 1, wherein the spike protein comprises a SARS-CoV-2 spike protein.

8. The 2-DG according to claim 1, wherein the viral infection has developed into Covid-19.

9. The 2-DG according to claim 1, wherein the 2-DG is provided as a micron or a submicron particle in the preparation, wherein said micron or submicron particle is a mechanically micronized particle or is a micronized particle obtained by spray drying, or the 2-DG is provided as the preparation in a liposomal or a proliposomal formulation.

10. The 2-DG according to claim 9, wherein the preparation comprises an amount of the 2-DG in a range of 1% to 75% w/w of a total weight of the preparation.

11. The 2-DG according to claim 10, wherein the preparation comprises one or more further excipients selected from the group consisting of:
an amino acid in an amount of 0% w/w up to 80% w/w of the total weight of the preparation;
trehalose in an amount of 0% w/w up to 60% w/w of the total weight of the preparation;
mannitol, 0% w/w up to 60% w/w of the total weight of the preparation;
propylene glycol or/and, glycerol, ethyl alcohol in a concentration range from 10% to 80% of the total weight of the preparation;
one or more further phospholipid, one or more further negatively or a positively charged phospholipid in an amount of 1% up to 10% of the molar % of the phospholipid fraction, wherein the one or more further phospholipid is selected from the group consisting of: phosphatidylglycerol, dimyristoyl phosphatidylglycerol, dipalmitoylphosphatidylglycerol, hydrogenated soybean phosphatidylcholine (HSPC), and soybean phosphatidylcholine (SPC);
sterol in an amount of 0 molar % up to 55 molar % of the total lipid fraction;
nicotinic acid amide, in an amount of 10% w/w up to 80% w/w of the total weight of the preparation; and
urea in an amount of 20% w/w up to 80% w/w of the total weight of the preparation.

12. The 2-DG according to claim 9, wherein the preparation further comprises an excipient comprising a lipid fraction comprising a phospholipid fraction in an amount of 5% to 80% w/w.

13. The 2-DG according to claim 9, wherein:
liposome sizes range from 30 nm to 200 nm for intravenous delivery;
liposome sizes range from 50 nm to 5 μm for pulmonary delivery;
unilamellar liposomes sizes range from 30 to 120 nm.

14. The 2-DG according to claim 9, wherein the amount of 2-DG in the liposomal or proliposomal formulation is in a range of approximately 10 mg to 1000 mg.

15. The 2-DG according to claim 1, wherein the 2-DG is provided as the preparation for administration by inhalation, wherein the preparation comprises particles for inhalation with a diameter of 10 μm or less.

16. A process for preparing a proliposome- or liposome-encapsulated pharmaceutical composition comprising 2-Deoxy-D-Glucose (2-DG) for use in a medical method to prevent and/or to treat a viral infection in a subject by a virus comprising a spike protein, wherein the 2-DG is provided as a preparation in an amount and a formulation to tissue of the subject that results in an effective tissue concentration to partially or completely inhibit glycosylation of the spike protein.

17. A method of manufacturing a proliposome- or liposome-encapsulated pharmaceutical composition comprising 2-Deoxy-D-Glucose (2-DG) for use in a medical method to prevent and/or to treat a viral infection in a subject by a virus comprising a spike protein, wherein the 2-DG is provided as a preparation in an amount and a formulation to tissue of the subject that results in an effective tissue concentration to partially or completely inhibit glycosylation of the spike protein.

18. (canceled)

19. A device for inhaling a substance in the form of aerosol particles or powder particles, the device comprising:
a discharge nozzle for dispensing the substance in the form of aerosol particles or powder particles;
a container for receiving and keeping the substance; and
an actuator for activating the device, the actuator being configured to release a certain amount or dose of the substance kept in the container for conveying the substance through the discharge nozzle of the device, wherein the aerosol particles or powder particles comprise 2-Deoxy-D-Glucose (2-DG).

20. The device of claim 19, wherein the substance comprises the 2-DG and at least one active ingredient selected from the group consisting of: ribavirin, emetine, and NMS-873.
